(19) United States
(12) Patent Application Publication
(10) Pub. No.: US 2022/0305248 A1
(43) Pub. Date: Sep. 29, 2022

(54) IMPLANTABLE VASCULAR ACCESS DEVICE

(71) Applicant: EMODIAL S.r.l., CASSANA (IT)

(72) Inventor: Gianni PECORARI, MIRANDOLA (MO) (IT)

(21) Appl. No.: 17/618,985

(22) PCT Filed: Jun. 17, 2020
(86) PCT No.: PCT/IB2020/055640

§ 371 Date: Dec. 14, 2021

(30) Foreign Application Priority Data
Jun. 18, 2019 (IT) 102019000009318
Jun. 5, 2020 (IT) 102020000013321

(51) Int. Cl. A61M 39/02; A61M 1/36; A61M 5/158

(52) U.S. Cl. CPC A61M 39/0247; A61M 1/3655; A61M 1/3661; A61M 5/158; A61M 2005/1587; A61M 2039/0223; A61M 2039/0258; A61M 2209/06

(57) ABSTRACT

A vascular access device for haemodialysis permanently implantable in a patient having a pre-existing arteriovenous fistula or, alternatively, in a vein, an artery or between a vein and an artery, to create an artificial arteriovenous fistula and a vascular access healing device and a sterile package containing a single-use device of collecting and/or injecting blood usable in combination with the vascular access device.

### FIELD OF THE INVENTION

The invention relates to the field of haemodialysis devices. In particular, the invention relates to a vascular access device for haemodialysis permanently implantable in a patient having a pre-existing arteriovenous fistula and a sterile package containing a single-use device of collecting and/or injecting blood from and to the patient utilisable in combination with a vascular access device for haemodialysis with which a collection and/or injection system for haemodialysis can be obtained which comprises both devices and which advantageously enables carrying out the surgical and haemodialysis methods described herein.

### DESCRIPTION OF THE PRIOR ART

Haemodialysis is a treatment that enables, by means of extracorporeal circulation of blood, purifying the blood in a special haemodialysis apparatus which comprises a semi-permeable membrane through which only (or at least the majority of) the toxic substances that are to be removed pass. Patients suffering from severe renal failure are subject to this treatment. Obviously, in order to carry out the haemodialysis it is necessary to create a vascular access, i.e. two points: one from which the blood to be purified is to be collected, and the other from which the filtered blood is re-injected into the circulatory system. The vascular access most generally used is the arteriovenous fistula (also known as a FAV) which is realised by a surgical operation, creating a permanent connection between a vein and an artery which involves the widening of the portion of vein of the arteriovenous fistula as the arterial pressure is much higher than the venous pressure. Therefore the two above-mentioned points of the vascular access can be predisposed in the arteriovenous fistula, as this enables passage of the relatively high blood flows, and reduces the time necessary for the haemodialysis. Notwithstanding this, haemodialysis is a long treatment, and in fact requires from three to four hours and must be carried out two or three times a week. Consequently the arteriovenous fistula is periodically and frequently subjected to the traumas deriving from the insertion of metal needles, known as fistula needles, which are made of metal, have a diameter in the order of 1.6 mm, as well as a sharp and cutting tip. The needles are to be inserted in the arteriovenous fistula and can create traumas to the portion of wall of the FAV opposite the inlet portion, in particular because in order to insert in the vein the fistula needle is inserted with a first inclination with respect to the skin and to insert it in the FAV it is necessary to change that inclination and position the needle parallel to the fistula. This causes stress, pain and traumas to the patient (the bruising alone is testimony to this), the risk of the FAV passing completely from side to side, complications for individuals who suffer from vein fragility and a high possibility of infection. In fact, even when administering local anaesthesia, the patient, once the effect of the anaesthesia has faded, still feels pain. To obviate this, the “Buttonhole” method has been developed, which includes inserting needles in the FAV always at the same two points (collection and return) with the same angulation and depth with the purpose of creating a sort of subcutaneous channel of scar tissue that extends from the cutaneous surface to the FAV through which the needles are inserted prior to every haemodialysis session. Special devices have also been created which are to be inserted into the channel from session to session in order to keep it open until the channel has been stably formed. It is clear that it is of fundamental importance to guarantee the same angulation and the same depth during each insertion of the needle. On this matter, first the above-mentioned fistula needles (sharp and cutting) have to be used for a certain number of dialysis sessions, and only after this can needles be used that are suitable for the Buttonhole method, which needles have the same diameter as the fistula and a bevelled tip which does not cut. Therefore highly specialised personnel is necessary for practising the method, especially in the early days of treatment. Further, the Buttonhole method is correlated to a greater incidence of infections with respect to the use of cutting needles and further it is not recommended in a case in which the subcutaneous tissue is too much or too little and where there is scarred skin. Recently vascular access devices for haemodialysis have been developed which are permanently implantable in a patient having an arteriovenous fistula. These devices have a fixing surface destined to be fixed to the outer wall of the FAV proximal to the skin and which comprise a funneling channel opening into the thinnest part in the fixing surface and in which, once implanted, the device remains stuck in the muscle. With these devices too, it is necessary to use a fistula needle which is inserted into the skin and the muscle, seeking to centre the funneling channel and gradually, as it is being inserted, passes through the wall of the FAV at the fixing surface of the device. This device does not do away with the risk of crossing the vein (V) with the fistula needle from side to side: further, owing to the funneling channel, there is no possibility of a variety of inclinations of the fistula needle which therefore cannot be inserted deeply, nor does it enable a reduction of the treatment time which is limited by the section of the fistula needle, alike to the Buttonhole method.

Documents US 2010/318016 A1 and U.S. Pat. No. 4,822,341 describe further vascular access devices for haemodialysis permanently implantable in a patient having a pre-existing arteriovenous fistula. The device according to U.S. Pat. No. 4,822,341 is implanted in series to a blood vessel. Once implanted, the device has an inlet hole and an outlet hole, respectively for the collection and re-injection of blood. This device includes a sliding valve for closing and opening the holes by sliding longitudinally with respect to the blood vessel, and once the device has been implanted the sliding valve is arranged subcutaneously. It is clear that in order to open and close the holes, the operator must cause the sliding valve to slide internally of the patient's body to oppose the action of the tissues at the valve, and that the operator also has to use a fistula needle, inserting it into the skin and the muscle, seeking to centre the holes, once open. Owing to the objective difficulties in sliding the implanted valve in the body, the device has virtually never had a practical application. Therefore a need emerges to carry out the haemodialysis sessions while limiting the stress, pain, and traumas for the patient, the damage to the FAV, the possibility of infections of the vascular access sites of the FAV, the need for highly specialised personnel and the related costs. There additionally emerges a need to reduce the number of operations the patient has to be subjected to in order to prepare her or him for haemodialysis with the aim of reducing the risks correlated with the operations and the disturbance and pain caused to the patient. Further, there is an acknowledged need to reduce the costs and/or times for haemodialysis, as well as the need for specialised healthcare operators.

### SUMMARY OF THE INVENTION

The aim of the present invention is to obviate or limit the drawbacks correlated to haemodialysis using FAV. In particular, the main aim of the present invention is to provide a haemodialysis treatment while limiting the stress, pain, and traumas for the patient, the damage to the FAV or other blood vessels, the possibility of infections of the vascular access sites of the FAV, the need for highly specialised personnel and the related costs. Additionally, a further aim of the present invention is to realise a vascular access device which is constructionally simple and economical, as well as reliable in use. A further aim of the present invention is to enable a reduction of haemodialysis times. The above-mentioned aims are attained according to the contents of independent claims 1, 2, 10 and 13. The vascular access device for haemodialysis of claim 1 can be permanently implanted in series to the pre-existing FAV, to the vein or artery or can hydraulically connect a vein to an artery. The sterile package containing at least a single-use device of collecting and/or injecting blood in an arteriovenous fistula as defined in claim 14 can be opened and used in each haemodialysis session hydraulically connecting the connecting element of the single-use device to an inlet tube of blood or to an outlet tube of blood of a haemodialysis apparatus, by acting on the flow regulating and/or intercepting device to enable the flow of the cannula and inserting the first end of the medical cannula (possibly piercing, with a needle, the patient's skin only at the second end of the second tubular element of the second conduit) through the second and first conduit of the vascular access device implanted according to the invention. As the only body element that is pierced is the skin, which easily heals, in particular if treated with appropriate fistula pressure pads comprising silver and/or another disinfecting agent and/or a healing agent, the pain caused to the patient during the haemodialysis sessions is significantly reduced. It is further clear that, according to the invention, there is never a risk of passing from side to side of the vein, artery or arteriovenous fistula during the haemodialysis sessions as the insertion of the collecting or return device from and into the vein is achieved by cannulation and takes place at the vascular access device and with the inserting of a free end of a medical cannula for cannulation. This end is by definition non-cutting and is sufficiently flexible to conform and insert, first in the second conduit and then in the first conduit of the vascular access device for haemodialysis. Consequently there is no risk of damage to the FAV. Additionally, the first conduit can have the same internal diameter as the FAV, the vein or the artery and the cannula can have, as a function of the relative thickness, a diameter of less by 0.1-0.2 mm to the internal diameter of the first conduit, i.e. an internal diameter of 2.0-4.0 mm, therefore much greater than the diameter of the fistula needle, which is 1.6 mm. This enables notably increasing the quantity of blood collected and returned into the FAV during haemodialysis. Therefore, by appropriately dimensioning the filtering surface of the semi-permeable membrane of the haemodialysis apparatus on the basis of the blood flow that can flow into the cannula having the internal diameter of 2.0-4.0 mm, it will be possible to significantly limit the times and costs of personnel relative to a single haemodialysis treatment while increasing personnel productivity.

Note that the use of the vascular access healing device of the invention enables, by inserting it at the point of vascular access of the body and in the second conduit and/or in the third conduit, healing device the valve access about the cylinder of the healing device. In fact, by leaving the healing device for a few days in the site, removing it for periodic medication of the vascular access (at least 2-3 times a week), and replacing it, after each medication, it is possible to obtain, in a relatively short time, a permanent vascular access having a permanently open healed vascular access hole. The vascular access hole opens on the second conduit and/or on the third conduit of the vascular access device of the invention to which is preferably connected, respectively, the first or second valve, which hydraulically close the respective second or third conduit. In this way blood loss can be drastically limited. Note that the permanent vascular access having a healed access hole is passed through by a cannula for cannulation, in particular the cannula comprised in the sterile package of the invention. It is therefore not necessary to use fistula needles to carry out the dialysis. Consequently the dialysis can also be carried out by an operator who is not highly specialised in dialysis as the inlet of the cannula in the permanent vascular access is enormously simplified with respect to the prior art and further facilitates the connection of the haemodialysis apparatus to the patient. Further, as there is no use of fistula needles, the discomfort and pain felt by the patient is reduced, as is the probability of damaging veins, arteries or the FAV.

According to the characteristics of the person to be dialysed, the time required for obtaining the permanent vascular access with the permanently open healed vascular access hole can vary from 4 to 12 weeks, or from 8 to 10 weeks. In this period and in any case, in a case where the healing device of the invention is not used, until the implant of the vascular access device has matured, (i.e. is ready to be used) dialysis is not carried out or is carried out using a central venous catheter, which will then be eliminated. Additionally to the advantages set out in the foregoing, the vascular access device for haemodialysis of claim 1 further and advantageously enables choosing the blood vessel in which to implant it; in fact it can be implanted in series to an artery (A) to a vein, to a pre-existing arteriovenous fistula or, more preferably, it can be implanted between an artery and a vein to constitute in itself an artificial arteriovenous fistula. In this case, with a single operation, a permanent vascular access and outlet and an artificial arteriovenous fistula are created, thus limiting the number of operations to which the patient has to be subjected, as well as the consequent risks of post-operatory complications and the discomfort suffered by the patient.

### BRIEF DESCRIPTION OF THE DRAWINGS

The characteristics of the invention will emerge in the following, which describes relative preferred but non-exclusive embodiments, with reference to the appended tables of drawings, in which:

FIGS. 1A, 1B, 1C, 1D and 1E are schematic views, respectively perspective, lateral, from above, frontal, and in section with respect to plant IE-IE of FIG. 1D of a first embodiment of a vascular access device for haemodialysis according to the invention;

FIGS. 2A, 2B, 2C, 2D and 2E are schematic views, respectively perspective, lateral, from above, frontal, in section with respect to plant 2E-2E of FIG. 2D of a second embodiment of a vascular access device for haemodialysis according to the invention;

FIG. 3 is a larger-scale view of detail K of FIG. 2B;

FIG. 4 is a schematic perspective view of a third embodiment of a vascular access device for haemodialysis according to the invention;

FIG. 5A is a view from above of a sterile package according to the invention; FIG. 5B is a lateral view of a further embodiment of the single-use device of collecting and/or injecting blood according to the invention; FIG. 5C is a view from above of the single-use device of FIG. 5B; FIG. 5D is a section view according to axis V_{D}-V_{D} of FIG. 5C of the single-use device of FIG. 5B; FIG. 5E is a front view of the single-use device of FIG. 5B; FIG. 5F is a larger-scale view of detail A of FIG. 5D; FIG. 5G is a perspective view of the single-use device of FIG. 5B; FIG. 5I is a larger-scale view of detail B of FIG. 5G; FIG. 5H is a larger-scale view of detail C of FIG. 5G; FIG. 5L is a lateral view of a further embodiment single-use of the single-use device for collecting and/or injecting blood; FIG. 5M is a view from above of the single-use device of FIG. 5L; FIG. 5N is a section view according to axis V_{N}-V_{N} of FIG. 5M of the single-use device of FIG. 5L; FIG. 5O is a front view of the single-use device of FIG. 5L; FIG. 5P is a larger-scale view of detail D of FIG. 5N; FIG. 5Q is a perspective view of the single-use device of FIG. 5L; FIG. 5R is a larger-scale view of detail D of FIG. 5G; FIG. 5S is a larger-scale view of detail D of FIG. 5G;

FIG. 6 is a schematic view in section of a first embodiment of a portion of a collection and/or injection system for haemodialysis according to the invention coupled to an arteriovenous fistula in a use configuration;

FIG. 7 is a schematic view in section of a second embodiment of a portion of a collection and/or injection system for haemodialysis according to the invention coupled to an arteriovenous fistula in a use configuration;

FIG. 8 is a schematic view in section of a third embodiment of a portion of a collection and/or injection system for haemodialysis according to the invention coupled to an arteriovenous fistula in a use configuration;

FIG. 9A is a lateral view of a fourth embodiment of a vascular access device according to the invention; FIG. 9B is a rear view of the device of FIG. 9A, FIG. 9C is a view from above of the device of FIG. 9A, FIG. 9D is a section view of the device of FIG. 9A along plane IX_{D}-IX_{D} of FIG. 9C, FIG. 9E is a section view of the device of FIG. 9 along plane IX_{E}-IX_{E} of FIG. 9B, FIG. 9F is a larger-scale view of detail J of FIG. 9D; FIG. 9G is a section view of the device of FIG. 9A according to plane IX_{G}-IX_{G} of FIG. 9O, FIG. 9H is a section view of the device of FIG. 9A according to plane IX_{H}-IX_{H} of FIG. 9G, FIG. 9I is a larger-scale view of detail K of FIG. 9H, FIGS. 9L and 9M are perspective views, respectively from below and above, of the device of FIG. 9A,

FIG. 10A is an exploded view from above of the device of FIG. 9A, FIG. 10B is a rear view of the exploded device of FIG. 10A, FIG. 10C is a section view of the exploded device of FIG. 10A according to plane X_{C}-X_{C} of FIG. 10A, FIG. 10D is a section view of the exploded device of FIG. 10A according to plane X_{D}-X_{D} of FIG. 10B, FIG. 10E is a perspective view of the exploded device of FIG. 10A, FIG. 10F is a larger-scale view of detail X of FIG. 10D,

FIG. 11A is a lateral view of a fifth embodiment of a vascular access device for haemodialysis according to the invention, FIG. 11B is a rear view of the device of FIG. 11A, FIG. 11C is a view from above of the device of FIG. 11A, FIG. 11D is a section view of the device of FIG. 11A according to plane XI_{D}-XI_{D} of FIG. 11C, FIG. 11E is a section view of the device of FIG. 11A according to plane XI_{E}-XI_{E} of FIG. 11B, FIG. 11F is a larger-scale view of detail K of FIG. 11D, FIG. 11G is a section view of the device of FIG. 11A according to plane XI_{G}-XI_{G} of FIG. 11O, FIGS. 11L and 11M are perspective views, respectively from below and above, of the device of FIG. 11A,

FIG. 12A is an exploded view of the device of FIG. 11A, FIG. 12B is a rear view of the exploded device of FIG. 12A, FIG. 12C is a section view of the exploded device of FIG. 12A according to plane XII_{C}-XII_{C} of FIG. 12A, FIG. 12D is a section view of the exploded device of FIG. 12A according to plane XII_{D}-XII_{D} of FIG. 12B, FIG. 12E is a perspective view of the exploded device of FIG. 12A, FIG. 12F is a larger-scale view of detail K of FIG. 12D,

FIG. 13A is a lateral view of a sixth embodiment of a vascular access device according to the invention, FIG. 13B is a rear view of the device of FIG. 13A, FIG. 13C is a view from above of the device of FIG. 13A, FIG. 13D is a section view of the device of FIG. 13A according to plane XIII_{D}-XIII_{D} of FIG. 13C, FIG. 13E is a section view of the device of FIG. 13A along plane XIII_{E}-XIII_{E} of FIG. 13D, FIG. 13F is a larger-scale view of detail K of FIG. 13D, FIGS. 13G and 13H are perspective views, respectively from below and above, of the device of FIG. 13A,

FIG. 14A is an exploded view of the device of FIG. 13A, FIG. 14B is a rear view of the exploded device of FIG. 14A, FIG. 14C is a section view of the exploded device of FIG. 14A according to plane XIV_{C}-XIV_{C} of FIG. 14A, FIG. 14D is a section view of the device of FIG. 14A according to plane XIV_{D}-XIV_{D} of FIG. 14B, FIG. 14E is a larger-scale view of detail Z of FIG. 14D, FIG. 14F is a perspective view of the device of FIG. 14A,

FIGS. 15A, 15B, and 15C are, respectively, lateral and perspective views from below and above of a seventh embodiment of a vascular access device according to the invention

FIGS. 16A, 16B, and 16C are, respectively, lateral and perspective views from below and above of an eighth embodiment of a vascular access device according to the invention,

FIGS. 17A, 17B, 17C, and 17D are, respectively, lateral, longitudinal section and perspective views from below and above of a ninth embodiment of a vascular access device according to the invention;

FIG. 18A is a lateral view of a tenth embodiment of a vascular access device according to the invention; FIG. 18B is a rear view of the device of FIG. 18A, FIG. 18C is a view from above of the device of FIG. 18A, FIG. 18D is a section view of the device of FIG. 18A according to plane XIIX_{D}-XIIX_{D} of FIG. 18B, FIG. 18E is a view of the device of FIG. 18A from side P, FIG. 18F is a section view of the device of FIG. 18A according to plane XIIX_{F}-XIIX_{F} of FIG. 18A, FIG. 18G is a larger-scale view of detail J of FIG. 18D, FIG. 18H is a section view of the device of FIG. 18A according to plane XIIX_{H}-XIIX_{H} of FIG. 18A, FIG. 18I is a section view of the device of FIG. 18A according to plane XIIX_{I}-XIIX_{I} of FIG. 18H, FIGS. 18N and 18M are perspective views, respectively from below and above, of the device of FIG. 18A,

FIG. 19A is a lateral view of an eleventh embodiment of a vascular access device according to the invention, FIG. 19B is a rear view of the device of FIG. 19A, FIG. 19C is a view from above of the device of FIG. 19A, FIG. 19D is a section view of the device of FIG. 19A according to plane XIX_{D}-XIX_{D} of FIG. 19B, FIG. 19F is a larger-scale view of detail B of FIG. 19D, FIG. 19E is a section view of the device of FIG. 19A according to plane XIX_{E}-XIX_{E} of FIG. 19B, FIG. 19G is a larger-scale view of detail C of FIG. 19E, FIG. 19H is a section view of the device of FIG. 19A according to plane XIX_{H}-XIX_{H} of FIG. 19A, FIG. 19I is a section view of the device of FIG. 19A according to plane XIIX_{I}-XIIX_{I} of FIG. 19H, FIGS. 19N and 19M are perspective views, respectively from below and above, of the device of FIG. 19A;

FIGS. 20A, 20B, and 20C are views illustrating the positions in which a vascular access device according to the invention can be implanted,

FIGS. 21A, 21B, 21C, and 21D are schematic views, respectively, frontal, lateral, from above and perspective, of a healing device for valve access according to the invention in first relative configuration thereof and FIGS. 21E, 21F and 21G are schematic views, respectively, lateral, frontal and perspective, of the device of FIG. 2A in a second relative configuration.

### DESCRIPTION OF PREFERRED EMBODIMENTS

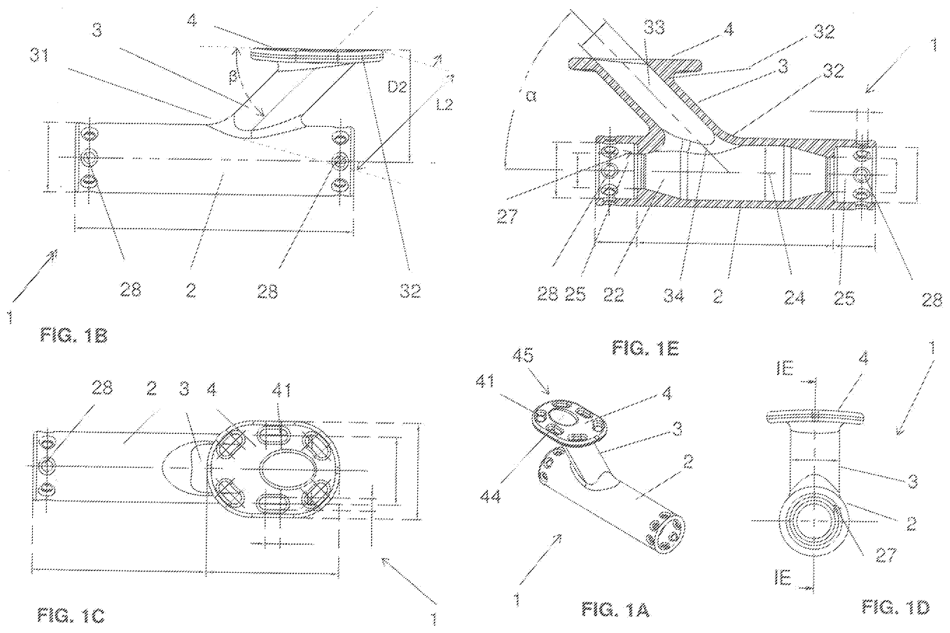

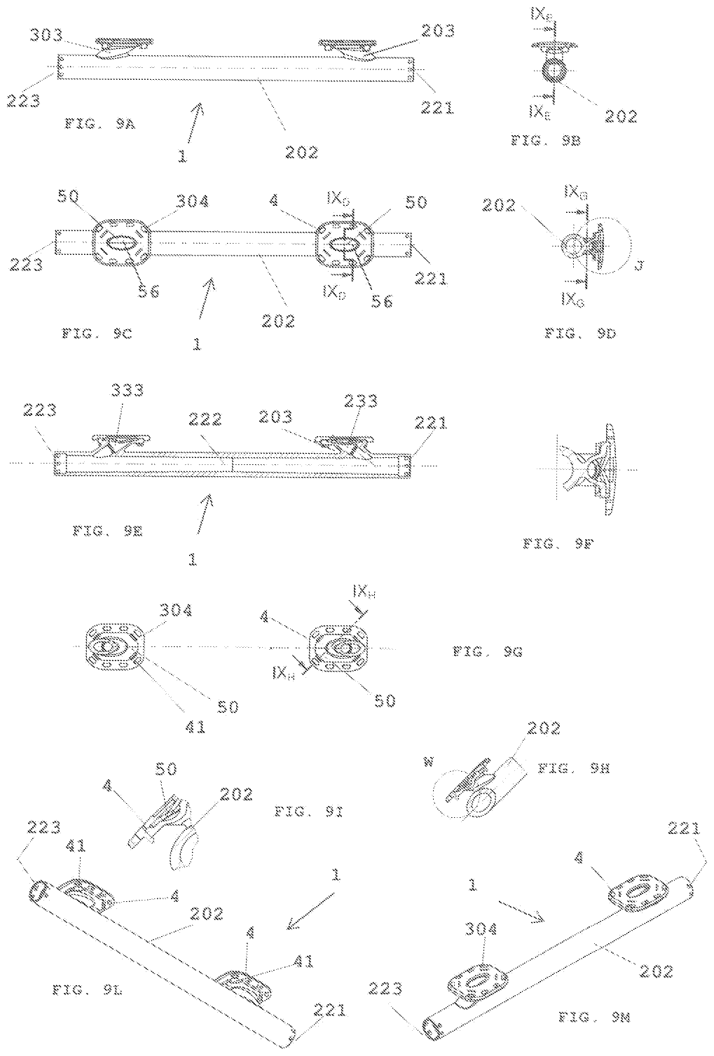

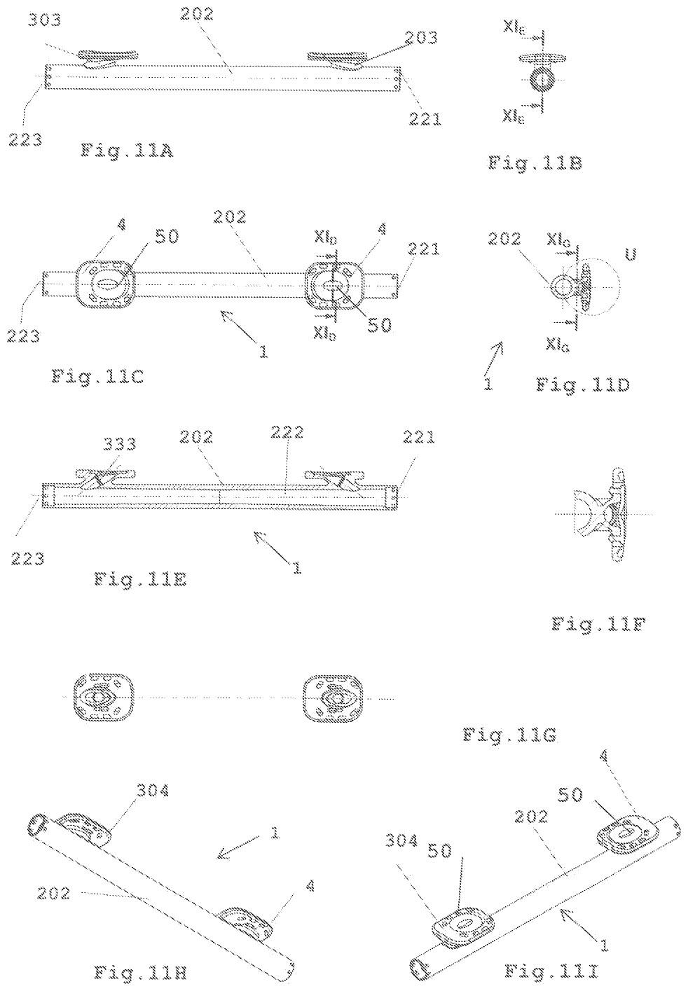

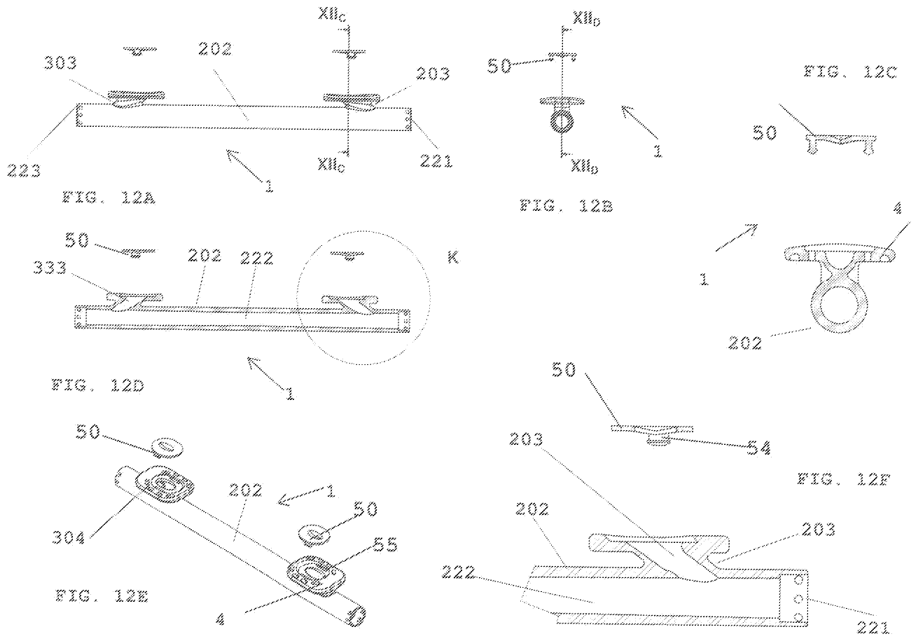

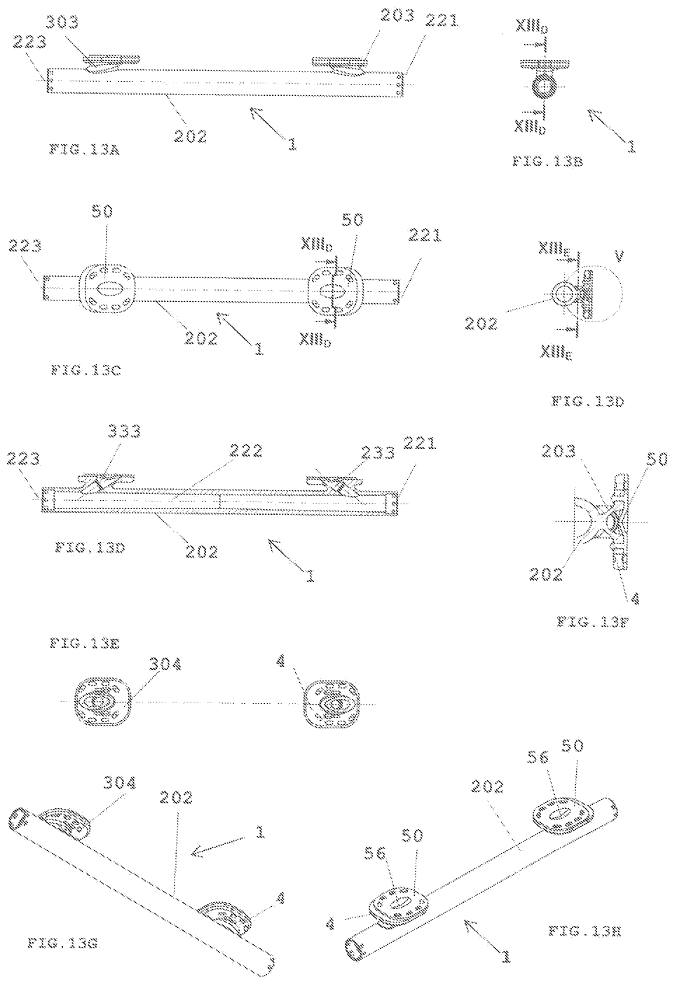

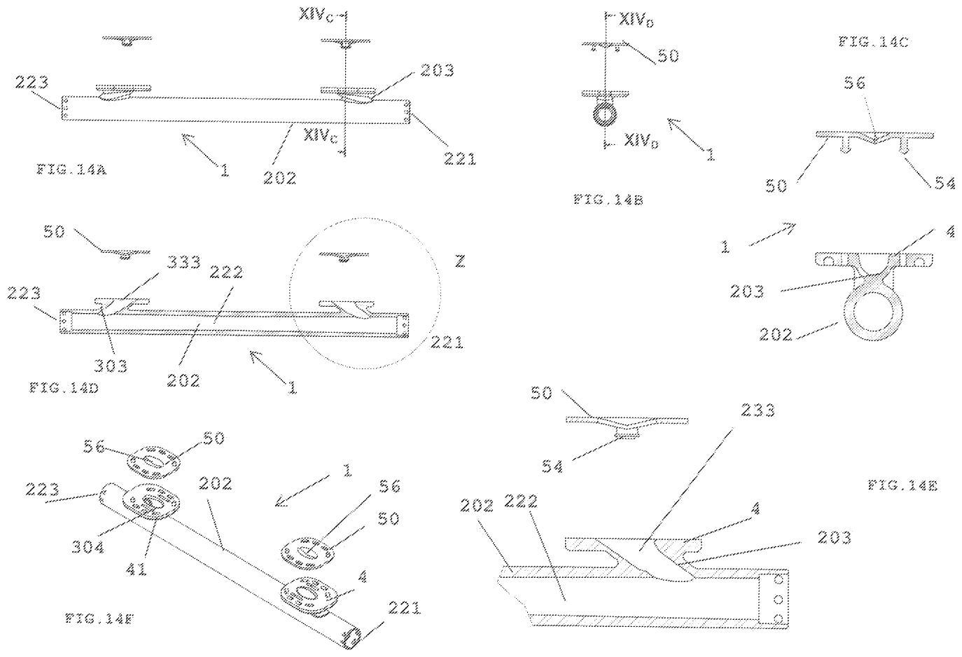

With reference to the figures, reference numeral (1) relates to a vascular access device for haemodialysis, permanently implantable in a patient having an arteriovenous fistula, (1) denotes a vascular access device for haemodialysis, (with both single and double vascular access) permanently implantable in a patient, (102) relates to a single-use device of collecting and/or injecting blood, (500) denotes a healing device for valve access, (100) a sterile package and (1000) denotes a collection and/or injection system for haemodialysis according to the invention. As can be noted from the figures, the embodiments, from the first to the third, relate to a vascular access device according to the single access invention (see FIGS. 1A-4 and 6-8), while the embodiments from the fourth to the eleventh relate to a vascular access device according to the invention which is double access as they enable a double vascular access (see FIGS. 9-19N). The vascular access device of the double access invention can in fact be used both for the collection, from the patient, of the blood to be subjected to haemodialysis, and for the re-injection into the patient of the blood subjected to dialysis for example by collecting the blood from the second tubular element (203) and re-injecting the blood in the third tubular element (303), or vice versa. The single access vascular access devices, however, enable only one access, either in inlet or outlet, and therefore two of them have to be implanted for each patient.

According to the invention, the single access vascular access device (1) is a vascular access device (1) made of a biocompatible and sterilisable material, and comprises:
a relative first tubular element (2) having a first and a second relative end (21) which are longitudinal, the first tubular element (2) defining a first conduit (22) which extends from the first to the second end (21);
a relative second tubular element (3) having a first and a second relative end (31, 32) which are longitudinal, and defining a second conduit (33) which extends from the relative first to the second end (31, 32); wherein the first end (31) of the second tubular element (3) is fixed to the first tubular element (2) in an intermediate position between the first and the second end (21) of the second tubular element (3), (see FIGS. 1A, 1B, 1E, 2A, 2B, 2E, and 4), so that the second conduit (33) is in fluid communication with the first conduit (22) and so that the second end (32) of the second tubular element (3) is on a first side with respect to the first tubular element (2);
first fixing means (4) comprising a first plurality of through-holes (41) and being fixed to the first tubular element (202) and/or second tubular element (3) (preferably they are fixed to the second tubular element (3)) the first plurality of through-holes (41) being arranged on the first side, with respect to the first tubular element (2), and at the second end (32) of the second tubular element (3), wherein said vascular access device (1) is conformed and dimensioned to be implantable in an arm of a patient having an arteriovenous fistula for haemodialysis with the first conduit (22) interposed in series to the arteriovenous fistula, with respect to the blood flow therein, between a first portion (51) and a second portion (52) of the arteriovenous fistula, the first end (21) and the second end (21) of the first tubular element (2) being fixed, respectively to the first portion (51) and to the second portion (52) of the arteriovenous fistula, at a relative end of the relative portion (51, 52) and the second end (32) of the second tubular element (3) and with the first plurality of through-holes (41) arranged subcutaneously at an implant skin region (7) in order to enable fixing of the first plurality of through-holes (41) to the implant skin region (7) by means of a first plurality of suture stitches (60), and a consequent fixing of the second end (32) of the second conduit (33) to the implant skin region (7). Note that, for the second conduit (33) to be in fluid communication with the first conduit (22) and so that the second end (32) of the second tubular element (3) is on a first side with respect to the first tubular element (2), the first end (31) of the second tubular element (3) cannot be fixed to the first tubular element (2) in an intermediate position between the first and the second end (21) of the second tubular element (3). In fact, the first end (31) of the second tubular element (3) is fixed in an intermediate position between the first and the second end (21) of the first tubular element (2) as can be observed in FIGS. 1A, 1B, 1E, 2A, 2B, 2E and 4.

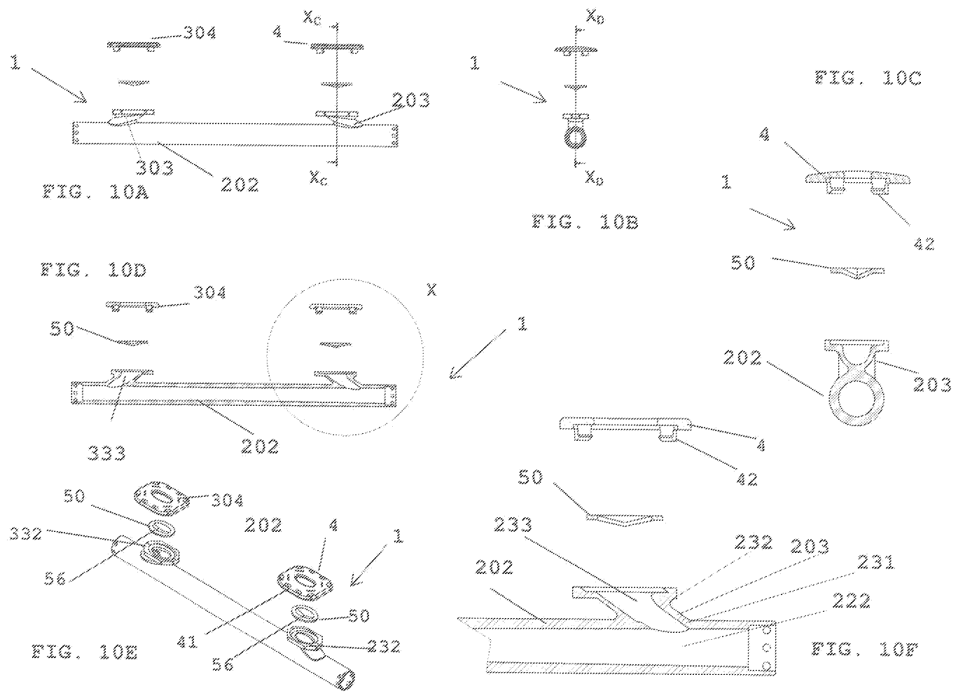

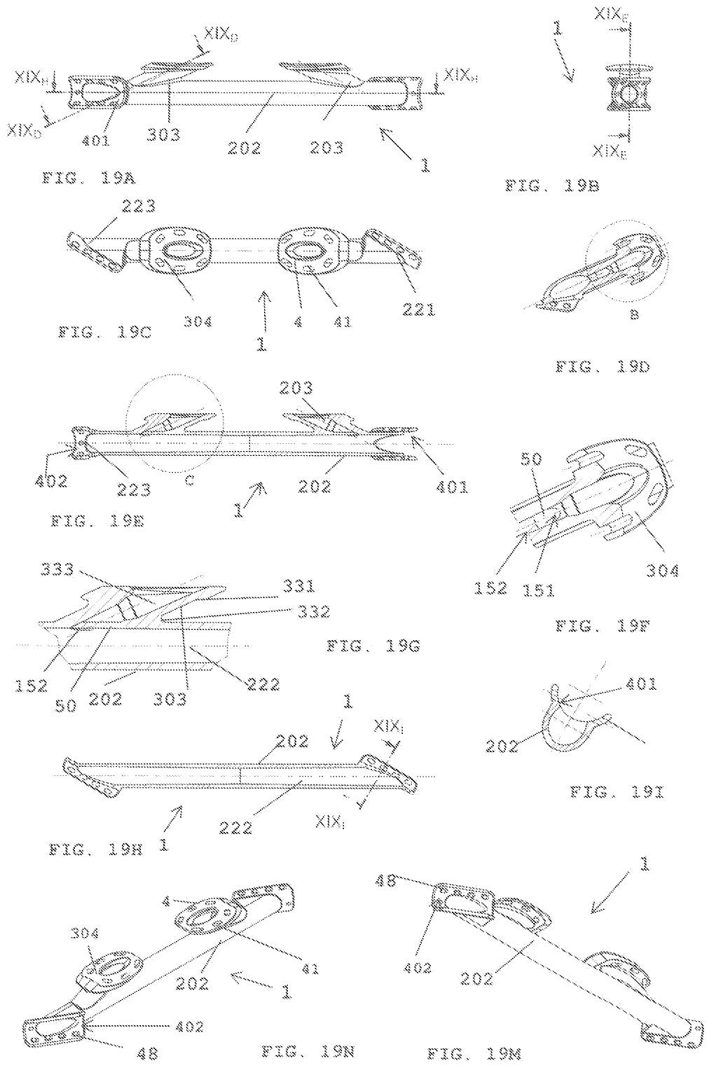

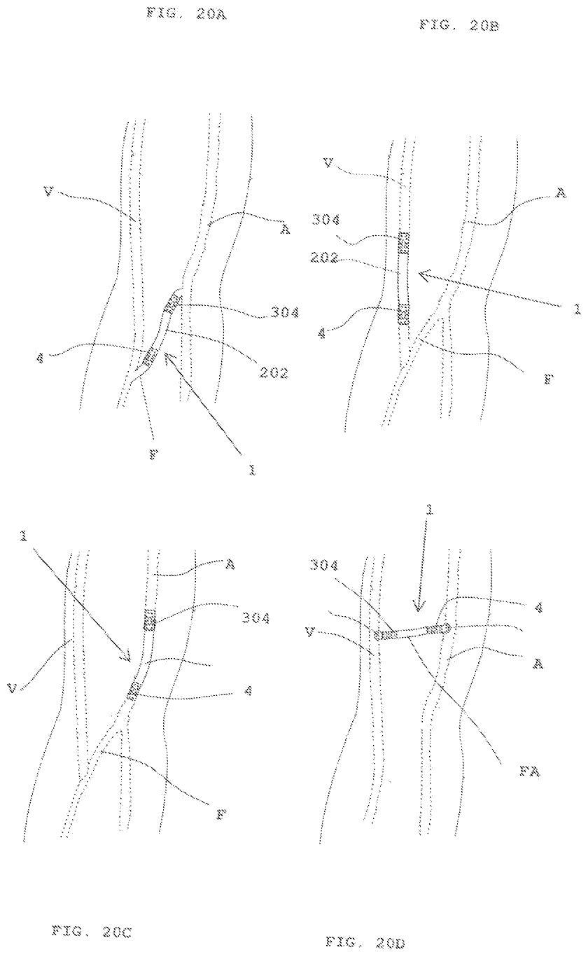

The vascular access device (1) of the invention, of the double access type, like those illustrated in FIGS. 9-19, is a vascular access device (1) for haemodialysis permanently implantable in a patient, and is made of a biocompatible and sterilisable material, and comprises:
a relative first tubular element (202) having a first and a second relative end (221, 223) which are longitudinal, the first tubular element (2) defining a first conduit (222) which extends from the first to the second end (221, 223);
a relative second tubular element (203) having a first and a second relative end (231, 232) (See FIG. 10F), which are longitudinal, and define a second conduit (233) which extends from the relative first to the second end (231, 232); wherein the first end (231) of the second tubular element (203) is fixed to the first tubular element (202) in proximity of the first end (221) of the first tubular element (202), so that the second conduit (233) is in fluid communication with the first conduit (222) and so that the second end (232) of the second tubular element (203) is on a first side with respect to the first tubular element (202);
a relative third tubular element (303) having a first and a second relative end (331, 332), which are longitudinal, the relative third tubular element (303) defining a third conduit (333) which extends from the relative first to the second end (331, 332) (See FIG. 19G); wherein the first end (331) of the third tubular element (303) is fixed to the first tubular element (202) in proximity of the second end (223) of the first tubular element (202), so that the third conduit (333) is in fluid communication with the first conduit (222) and with the second conduit (233) and so that the second end (332) of the third tubular element (303) is on the first side with respect to the first tubular element (202); and
first fixing means (4) comprising a first plurality of through-holes (41) and being fixed to the first tubular element (202) and/or second tubular element (203) the first plurality of through-holes (41) being arranged on the first side, with respect to the first tubular element (202), and at the second end (232) of the second tubular element (203),
second fixing means (304) comprising a second plurality of through-holes (41) and being fixed to the first tubular element (202) and/or third tubular element (303) the second plurality of through-holes (41) being arranged on the first side, with respect to the first tubular element (202), and at the second end (332) of the third tubular element (303), wherein the vascular access device (1) is conformed and dimensioned to be implanted in a patient, preferably a patient's arm, in:
a first position which is at an artery (A), at a vein (V), or at a pre-existing arteriovenous fistula (F) (see respectively FIGS. 20C, 20B and 20A) the first conduit being interposed in series with respect to the blood flow, respectively in the artery (A), in the vein (V) or in the pre-existing arteriovenous fistula (F) between a first portion and a second portion, respectively, of the artery (A), vein (V), or pre-existing arteriovenous fistula (F), with the first end and the second end (221, 223) of the first tubular element (202) fixed respectively to the first portion (51) and second portion (52), and/or
in a second position, between the artery (A) and the vein (V) (see FIG. 20D), with the relative first or second end (231, 232) of the first tubular element (202) fixed to the artery (A) with the remaining of the first or second relative end (231, 232) of the first tubular element (202), which is not fixed to the artery (A), fixed to the vein (V) in such a way that the first conduit (222) hydraulically connects the artery (A) to the vein (V) to constitute an artificial arteriovenous fistula (FA), wherein, in the first and second position, the second end (232) of the second tubular element (203) and the second end (332) of the third tubular element (303), the first and second plurality of through-holes (41) arranged subcutaneously in an implant skin region (7) in order to enable fixing of the first and second plurality of through-holes (41) to the implant skin region (7) by means of a corresponding first and a corresponding second plurality of suture stitches (60), and the consequent fixing, respectively, of the second end (232) of the second conduit (233) and the second end (332) of the third tubular element (303) to the implant skin region (7).

The double access vascular access device is not only implantable in series to a blood vessel (vein, artery A and pre-existing arteriovenous fistula), but also advantageously enables carrying out the following surgical method for implanting the device which comprises following steps:

predisposing the double access vascular access device of the invention; carrying out a surgical incision on a wall of a vein (V) at an implantation site; carrying out a surgical incision on a wall of an artery (A) at an implantation site; hydraulically connecting the vein and the artery via a first conduit 222, which is hydraulically connected at the relative ends, respectively to a second conduit (233) and to a third conduit (333); fixing the relative first or second end (231, 232) of the first tubular element (202) fixed to the artery (A) at the relative surgical incision; and fixing, with the remaining of the first or second relative end (231, 232) of the first tubular element (202), which is not fixed to the artery (A) at the relative surgical incision, to the vein (V) in such a way that the first conduit (222) hydraulically connects the artery (A) to the vein (V) to constitute an artificial arteriovenous fistula (FA), arranging the second end (232) of the second tubular element (203) and the second end (332) of the third tubular element (303), the first and second plurality of through-holes (41) arranged subcutaneously in an implant skin region (7); and fixing the first and second plurality of through-holes (41) to the implant skin region (7) by means of a corresponding first and a corresponding second plurality of suture stitches (60).

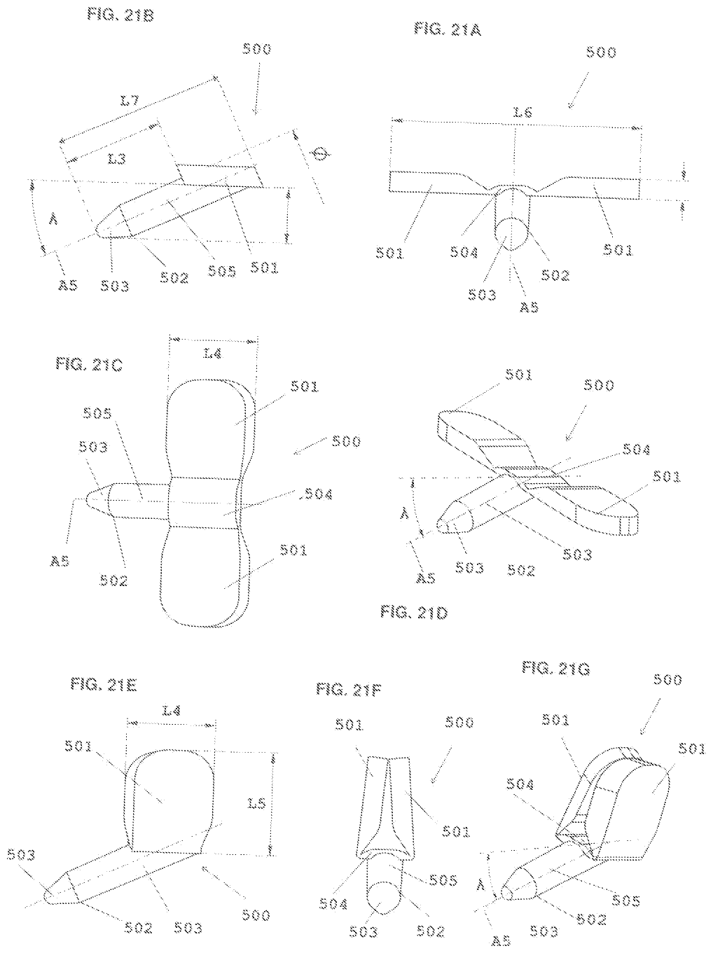

With the aim of obtaining a permanent vascular access having a permanently open healed vascular access hole the applicant has also developed a healing device (500) of a vascular access, comprising:
a relative insertion portion (502) which is insertable internally of a vascular access and which comprises: a cylinder (505) having a relative longitudinal development axis: a first and a second relative end which are longitudinal; and a distally tapered tip (503) (preferably coaxial to the cylinder) fixed to the first end of the cylinder (505); wherein the insertion portion is insertable in the second conduit (33, 233) or in the third conduit (333) of a vascular access device (1) for haemodialysis according to any one of claims 4, 7, 8, 9 and 10 from the relative second end (32, 232, 332) so as to occlude exclusively a portion of the second conduit (33, 233) or the third conduit (333) comprised between, respectively, the first valve (50) and the first plate (4) or between the second valve (50) and the second plate (304);
a relative connection portion (504) fixed to the second end of the cylinder (505); and
a relative first and a relative second gripping tab (501), fixed to the connection portion (504) on opposite sides thereof and on opposite sides with respect to the cylinder axis and with possibility of relative movement with respect to the connection portion (504), in order to be able to assume: a first relative configuration in which the first and the second gripping tab (501) are substantially coplanar (see FIGS. 21A-21D); and a second configuration in which the tabs are opposite one another (see FIGS. 21E-21F), wherein in both the first and the second configuration the first and the second gripping tab (501) are not insertable in the second conduit (33, 233) or third conduit (333).

In a relative preferred embodiment of the vascular access healing device, the relative cylinder is inclined with respect to the connection portion by an angle (A) (see respectively FIGS. 21B, 21D and 21G) identical to angle β (See FIG. 1A) with which the second tubular element (3, 203) or the third tubular element (303) is inclined with respect, respectively, to the first plate (4) or the second plate (304).

With reference to FIGS. 21A-21G, the length L3 of the insertable portion (502) is preferably comprised between 2 or 2.5 mm and 5 mm to avoid contacting the valve (50) it is preferably 1-3 mm. The gripping tabs (501) can have following dimensions: L4 comprised between 3 and 6 mm, preferably 4.5 mm and L5 comprised between 4.5 and 6 mm, preferably 5 mm. The diameter e of the cylinder obviously depends on the dimensions of the second and third conduit, but can advantageously be 1.5-2 mm, preferably 1.75 mm. The width L6 of the healing device (500) of the vascular access can be 10-15 mm, preferably about 12.5 mm, while the length L7 can be of 7-10 mm, preferably about 8.5 mm.

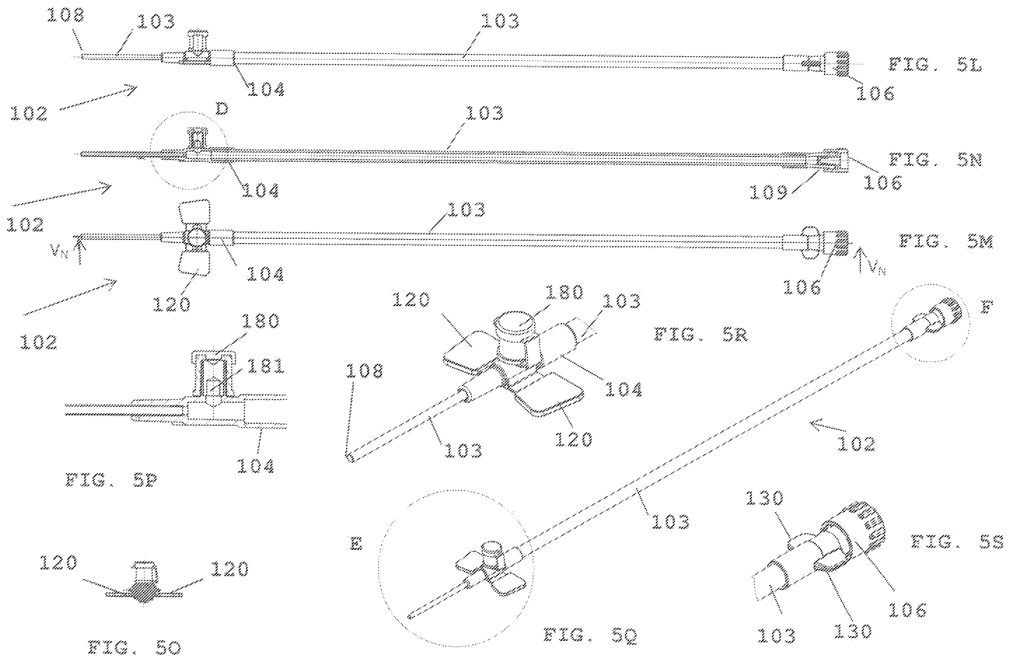

The sterile package (100) of the invention contains at least a single-use device (102) of collecting and/or injecting blood in an arteriovenous fistula constituted by:
a medical cannula (103) for cannulation having a relative first end (108) and a relative second end (109) wherein the first end (108) is free and cannulable in a pre-existing arteriovenous fistula (F), an artery (A), or a vein (V) of a patient through the second conduit (33, 233) and the first conduit (22, 222) or through the third conduit (333) and the first conduit (222) of both a double and single access device;
a flow regulating and/or intercepting device (104) predisposed to regulate and/or interrupt a flow of a liquid in the medical cannula (103); and
a connecting element (106), fixed to the second end (109) of the medical cannula (108) to enable hydraulic connection to an inlet tube of blood or to an outlet tube of blood of a haemodialysis apparatus;
optionally an access element arranged between the first end (108) of the cannula and the regulating and/or intercepting device (104) and defining an access channel (181) (see FIGS. 5F and 5P) hydraulically connected to the first end (108) of the cannula; and reversible closing means (180) for reversibly closing the access channel (181) at a distal end of the access element in order to enable injection of a liquid into the access channel (181) and to cause the liquid to flow towards the first end (108) of the medical cannula (103);
optionally a first gripping element (120), arranged between the first end (108) and/or the regulating and/or intercepting device (104), and/or a second gripping element (130), arranged proximally to the connecting element (106). Obviously the package also comprises a relative wrapping (101), sealed and made of a suitable material which enables sterilisation of the contents thereof, for example the wrapping (101) can be made with a sheet of a material that is permeable to ultra-violet rays and/or to ionising radiations (gamma and/or beta rays) and/or to microwaves. By appropriately selecting both the relative conformations and dimensions of the vascular access device (1) and the single-use device (102) of collecting and/or injecting, contained in the sterile package (100), it is sufficient to open the package at each haemodialysis session so as to easily obtain a collection and/or injection system (1000) for haemodialysis which comprises both a vascular access device (1) and the single-use device (102) of collecting and/or injecting, where the first free end (108) of the medical cannula (103) is free and cannulable in an arteriovenous fistula, a vein or an artery of a patient, through the second conduit (33, 233) and the first conduit (22, 222) and/or through the third conduit (333) and the first conduit (22, 222) of the vascular access device (1). The system 1000 enables attaining the above-mentioned aims, with respect to the prior art.

A collection and/or injection system (1000) for haemodialysis, according to the invention, thus comprises a vascular access device (1) for haemodialysis according to the invention which can be double or single access and at least a single-use device (102) of collecting and/or injecting blood in an arteriovenous fistula (F), a vein (V) or an artery (A) constituted by:
a medical cannula (103) for cannulation having a relative first end (108) and a relative second end (109) wherein the first end (108) is free and cannulable in a pre-existing arteriovenous fistula (F), an artery (A), or a vein (V) of a patient through the second conduit (33, 203) and the first conduit (22, 222) or through the third conduit (333) and the first conduit (22, 222) of a vascular access device (1);
a flow regulating and/or intercepting device (104) predisposed to regulate and/or interrupt a flow of a liquid in the medical cannula (103); and
a connecting element (106), fixed to the second end (109) of the medical cannula (108) to enable hydraulic connection to an inlet tube of blood or to an outlet tube of blood of a haemodialysis apparatus;
optionally an access element arranged between the first end (108) of the cannula and the regulating and/or intercepting device (104) and defining an access channel (181) (see FIGS. 5F and 5P) hydraulically connected to the first end (108) of the cannula; and reversible closing means (180) for reversibly closing the access channel (181) at a distal end of the access element in order to enable injection of a liquid into the access channel (181) and to cause the liquid to flow towards the first end (108) of the medical cannula (103); and
optionally a first gripping element (120), arranged between the first end (108) and/or the regulating and/or intercepting device (104), and/or a second gripping element (130) arranged proximally to the connecting element (106); and
wherein the collection and/or injection system (1000) can comprise optionally at least one healing device (500) of a vascular access according to the invention, preferably a plurality of the healing devices (500), and advantageously from eight to thirty.

In the case of the double access vascular access device it is preferable for the system to comprise two single access devices (102) for collecting and/or injecting.

Note that the access element and the closing means enable aspiration of any blood clots present in the access device, both single and double access. Additionally, they enable injecting an anticoagulant into the first conduit (22, 222). This anticoagulant can be heparin, for example heparin locks, sodium citrate, urokinase and mixtures thereof.

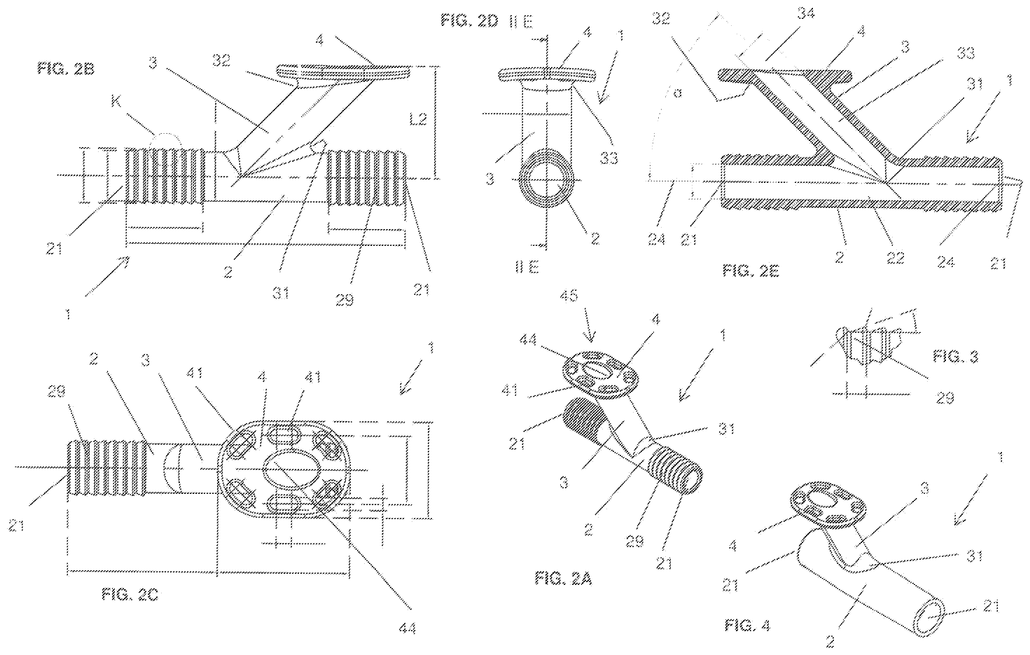

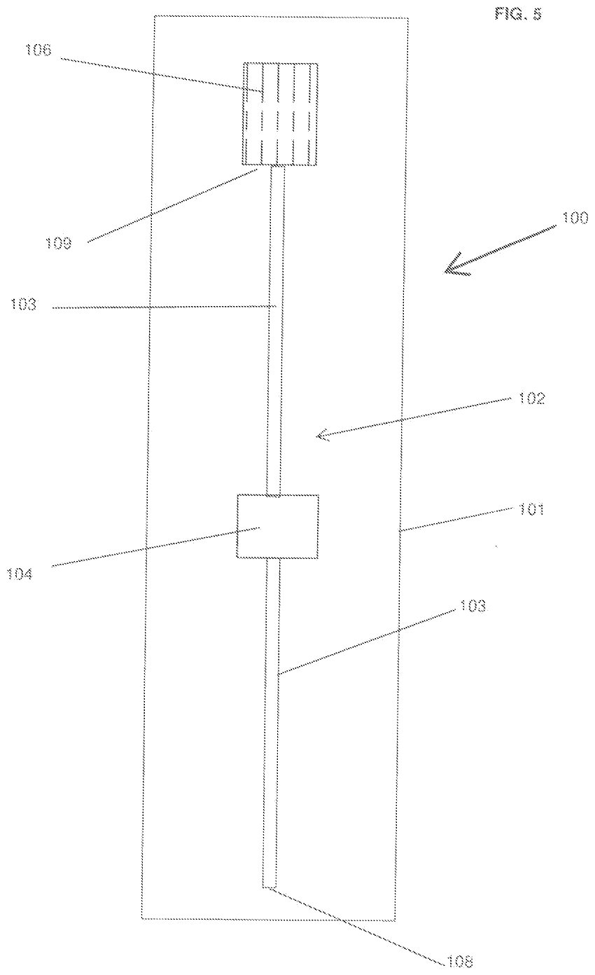

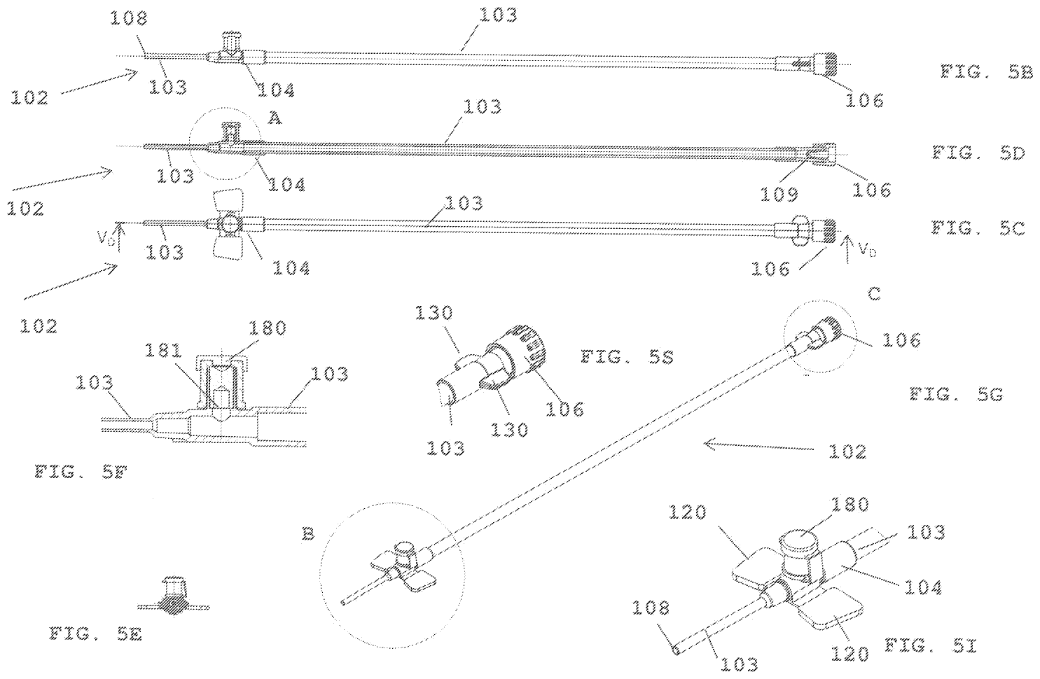

To improve the sterilisation and mechanical resistance of the vascular access device (1), it is preferably made of a metal, more preferably it is titanium, although the invention can include being constructed also using biocompatible and sterilisable plastic materials and not resorbable by the body, as described in the following. With the aim of improving the fixing of the second end (32, 232) of the second tubular element (3, 203) and the second end (332) with respect to the third tubular element (303), when present in the vascular access device for haemodialysis, to the skin region (7) for implant, the first and second plurality of through-holes (41) can be constituted by two through-holes (41), preferably arranged on the opposite side, respectively, to the second conduit (33, 232) and the conduit (333). In a case where it is constituted by three through-holes it is preferable to arrange them about the second conduit (33, 233) and the third conduit (333) at an angular distance of 120°, if a greater number of through-holes (41) is included, they can be arranged along a closed loop which surrounds the second conduit (33, 233) at the second end (32, 232) of the second tubular element (3, 203) (See FIGS. 1C and 2C). They can also preferably also be angularly equidistant. The through-holes (41) are preferably at least six in number. The same also applies to the holes of the second end (332) of the third tubular element (303), when the latter is present. To advantageously facilitate the insertion of the medical cannula (103) into the second (33) and the first conduit (22, 222), and/or into the first conduit (222) and into the third conduit (333) at least one of the two, between the cannula and the conduit, and preferably both, have relative oval internal sections, or, more preferably, circular. The conduits (22, 33) advantageously have cylindrical inner walls.

Obviously the first (2, 202) and second tubular element (3, 203) have relative longitudinal development axes (24, 34) which are incident to one another and define two angles of incidence, one supplementary to the other. This also applies to the first and third tubular element, in the case of the double access vascular access device. The preferred embodiments of the vascular access device (1) for haemodialysis are those in which the first (2) and second tubular element (3) have a relative longitudinal development axis incident to one another with a minimum angle (α) that is acute. This angle can preferably be comprised between 25° and 60°, more preferably between 25° and 50° and still more preferably between 28°-30° or 43°-48°, 25° and 45° and/or (preferably and) wherein, when the relative third tubular element (303) is present, the first tubular element (202) and third tubular element (303) have a relative longitudinal development axis incident to one another with a minimum angle (α) comprised between 25° and 50°, preferably 25°. The minimum angle (α) can advantageously be 25°, 35⁰ or 45°. Obviously, as a function of the angle (α) the length (L2) of the second tubular element (3) measured will have to be appropriately dimensioned, obviously along the relative longitudinal development axis, in order to dimension the vascular access device (1) so that by fixing the first tubular element (2) to the FAV, the second end (32) of the second tubular element (3) is under the skin (7). However, the surgeon, on the basis of the physical characteristics of the patient (gender, body type, muscle mass and fatty mass), will be perfectly able to choose a vascular access device (1) that is suitable for implanting in a patient, with the relative first tubular element (2) in series to the FAV and the second end (32) of the second tubular element (3) under the skin (7).

In an embodiment of the vascular access device (1) for haemodialysis of the invention, the first fixing means (4) can originate from the second end (32, 232) of the second tubular element (3, 203) and/or (preferably and), when present, the second fixing means (304) can originate from the second end (332) of the third tubular element (303), for example, the first and/or second fixing means (4) can be constituted by a plurality of arm braces, not illustrated, arranged spoke-fashion with respect to the second conduit (33), where each arm-band comprises one or more of the through-holes of the first plurality of through-holes (41).

It is particularly preferable for the first fixing means (4) to comprise a plate (4) having a relative main through-hole (44), wherein the first plurality of through-holes (41) is arranged peripherally with respect to the main through-hole (44), wherein the plate (4) is fixed at a relative first face to the second end (32) of the second tubular element (3, 203) so that the second conduit (33, 233) is accessible from the relative main through-hole and/or (preferably and), when present, for the second fixing means to comprise a second plate (304) having a relative main through-hole, wherein the second plurality of through-holes (41) is arranged peripherally with respect to the relative main through-hole and wherein the second further plate (304) is fixed at a relative first face to the second end (332) of the third tubular element (303) so that the third conduit (333) is accessible from the relative main through-hole (44). This simplifies the realisation of the vascular access device (1). The main through-hole (44) advantageously has the same section as the second conduit (33) at the second end and they coincide, which facilitates the insertion of the medical cannula (103) into the first conduit (22).

In an advantageous aspect of the invention, the plate (4) is fixed to the second tubular element (3) with a relative second face to the second end (45) opposite the relative first face and arranged along a plane that is parallel to the longitudinal development axis (24) of the first tubular element (2, 202) and/or (preferably and), when present, the second plate (304) is fixed to the third tubular element (303) by a relative second face (45) opposite the relative first face and arranged along a plane that is parallel to the longitudinal development axis (24, 224) of the first tubular element (2, 202). As illustrated in the embodiments of FIGS. 9-19 the second and third tubular element (203, 303), are preferably arranged in such a way that the relative longitudinal development axes cross from the first side of the first tubular element (202) as this facilitates blood collections to be subjected to dialysis and the return of the dialysed blood into the patient. The second face (44) is destined to internally face the skin (7) and is preferably arranged at a relative distance (D2), with respect to the longitudinal development axis (24) of the first tubular element (2, 202) comprised between 5 and 10 mm, preferably between 6.5 and 8.0 mm. This distance (D2) also obviously depends on the minimum angle (α) and the above considerations are valid relative to the dimensioning and the surgeon's choice regarding the vascular access device (1).

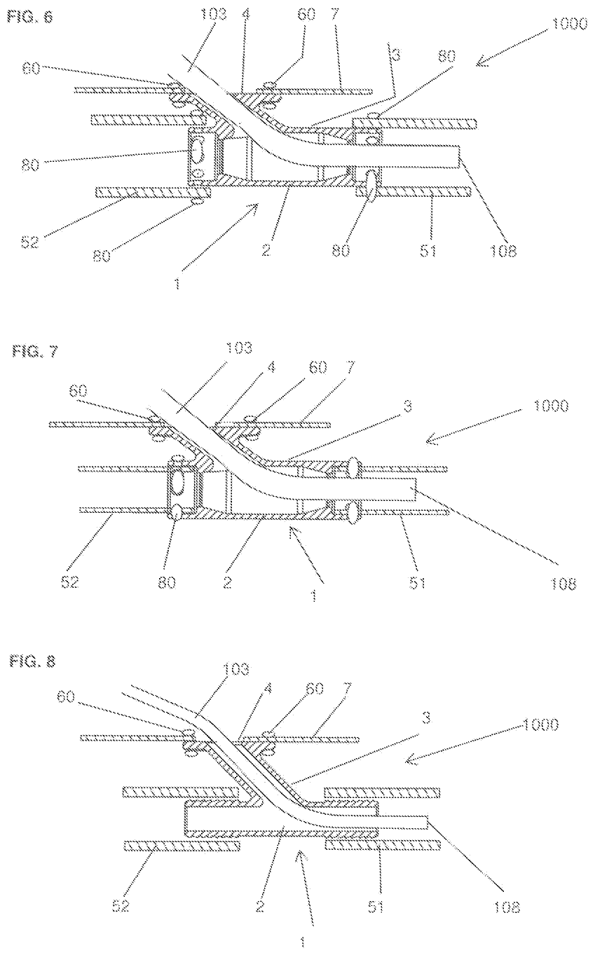

With the aim of easily fixing the vascular access device (1) for haemodialysis, both with single and double access, when the vascular access is to be permanently implanted in series to the pre-existing FAV, to the artery (A) or to the vein (V), the first end and the second end (21, 221, 223) of the first tubular element (2, 202) can advantageously each comprise a third plurality of though-holes (28) (see FIGS. 1A-1C, 6 and 16A-17D), arranged along a relative transversal fixing sector to enable fixing the first and the second end (21, 221, 223) of the first tubular element (2, 202) on an end, respectively, of the first portion (51) and the second portion (52) of the arteriovenous fistula by means of a third plurality of suture stitches (80) visible in FIG. 6. The suture can be carried out with the end of the first and second portion of artery (A), vein (V) or pre-existing arteriovenous fistula (F) arranged externally (as in FIG. 6) or internally of the first and/or second end of the first tubular portion. In this latter case, it is preferable that at least one from between the first end and the second end (21, 221, 223) of the first tubular element (2, 202), preferably both (as illustrated in FIG. 1E) each comprise a relative internal housing defined by the first conduit (22, 222) and comprising a relative internal annular abutment surface (27) for abutting (preferably entirely), when fitted on the end, respectively, of the first portion (51) and second portion (52) of the artery (A), the vein (V) or the pre-existing arteriovenous fistula (F), a terminal annular surface of the end, respectively, of the first portion (51) and the second portion (52) of the arteriovenous fistula (see FIGS. 7 and 20A) of the artery A (See FIG. 20C) or vein (See FIG. 20B). This not only improves the fixing but also prevents, once the free end (108) of the medical cannula (103) has been inserted in the second conduit (33) and in the first conduit (22), during the cannulation in a portion of the artery (A), the vein (V) or the pre-existing arteriovenous fistula (F) of the free end (108), the cannula from striking against the terminal annular surface of the end portion. Obviously if the housing (25) is present only in one from between the first end and the second end (21, 221, 223) of the first tubular element (2, 202), this will be the end opposite the minimum angle (α).

Preferably, the third plurality of through-holes (28) comprises at least 4, preferably at least 6 and more preferably at least 8. They are advantageously arranged angularly equidistant. The through-holes (41, 28) of the first and second and third plurality of though-holes must obviously have a dimension and a conformation such as to enable crossing by a surgical suture needle. During the suturing operation of the first plurality and second plurality of through-holes (28), respectively, to the skin (7) and to the two portions of the pre-existing FAV (F), of the vein (V) or the artery (A) cut by the surgeon, the surgeon will make each of the suture stitches pass through a different through-hole.

In a further fixing mode of the first and second end (21) of the first tubular element (2, 202) to the two portions of the pre-existing FAV (F), vein (V) or cut artery (A), the ends can be fixed to an end, respectively, of the first portion (51) and the second portion (52) of the pre-existing arteriovenous fistula of the vein (V) or the artery (A) using surgical glue (not illustrated).

For the purpose of improving the fixing with surgical glue of the first and/or the second end of the first tubular element (2, 202) each can comprise a relative transversal fixing sector having a relative irregular external surface, for example comprising a plurality of concavities, slots or grooves. In a preferred aspect of the invention the relative transversal fixing sector has a relative plurality of external annular grooves (29) (see FIGS. 2A-2E and 3) coaxial to the longitudinal development axis (24) of the first tubular element (2, 202) to improve the fixing by surgical glue of the first and second end (21) of the first tubular element (2, 202) to an end, respectively, of the first portion and the second portion of the pre-existing arteriovenous fistula of the vein (F) or the artery (A).

Considering the average body size of the patients, it is preferable for the vascular access device (1) for haemodialysis of the invention, single or double access, to have the following characteristics, taken alone or in combination:
the first conduit (22, 222) has a relative minimum internal radius comprised between 2 and 5 mm, preferably 2.5-4.0 mm, and/or a relative external radius comprised between 3 and 7 mm, 4 mm and 6.0 mm, preferably between 4 and 6.0 mm and/or
the second conduit (33, 233) and/or the third conduit (333), when present, has a relative minimum internal radius comprised between 2.0 and 5.0 mm, preferably 2.5-4 mm, and/or a relative external radius comprised between 3.0 and 6.0 mm, preferably between 3.5 and 5.0 mm and/or
the first plurality of through-holes (41) and/or the second plurality of through-holes (41), when present, is arranged at a distance comprised between 5-9 mm from the longitudinal development axis (24) of the first tubular element (2, 202), preferably 6.5-8 mm and also depends on the angle between the longitudinal development axes, and/or
the holes (41) of the first and/or second plurality of through-holes (41) have dimensions of di 1.1-2 mm, preferably 1.6×0.9, and/or
the holes of the third plurality of though-holes (28) have dimensions of 0.6-0.9 mm, preferably 0.7×0.85, and preferably 0.8.

It is particularly preferable, especially in the case of the double access vascular access device, for the length of the first tubular element to be 3-10 cm, more preferably 4-12 cm, 8-12 cm or advantageously 4-6 cm, both because in this way it is possible to implant it between a vein and an artery, in particular of an arm or a leg of a patient, and because in artificial conduits of this length it is possible for the same anatomic structure naturally present internally of the veins and the arteries to be recreated on the internal structure of the conduit. This significantly reduces the risk of blood clots and blocking of the first conduit (22, 222). Further, a length of the first tubular element (2, 202) of at least 4 cm, preferably 5-6 cm, ensures that the blood collected during dialysis does not contain blood just returned during dialysis. In a case where the vascular access device for haemodialysis is of the double access type, and is to be used to obtain an artificial arteriovenous fistula, the length is preferably at least 8-12 cm, which obviously depends on the patient's physical structure. Additionally, it is highly preferable for the device, or at least the inner walls of the first, second and third conduits (22, 222, 33, 233, 333), to be made of metal or biocompatible plastic materials. In the second case the device can be made by moulding or 3D printing. The preferred materials for making the device, especially if double access, are silicones, polymethyloxanes PDMSs, PTFE preferably ePTFE, DACRON, preferably silicones and/or polymethyloxanes and ePTFE, more preferably ePTFE, as they have a low propensity to thrombogenesis and enable a better endothelialisation of the relative first conduit (22, 220) which prevents the access device from becoming occluded; the risk of rejection is also reduced.

A vascular access device (1) for haemodialysis according to the invention is preferred further comprising a first valve (50) connected to the second conduit (33, 233) and configured so as, in a relative closed configuration, to hydraulically close the second conduit (33, 233) and, in a relative open configuration, to hydraulically open the second conduit (33, 233). In particular, in the case of a double access vascular access device, which therefore comprises the relative third tubular element (303), it is advantageous for it to also comprise a second valve (50) connected to the third conduit (333) and configured so as, in a relative closed configuration, to hydraulically close the third conduit (333) and so as, in a relative open configuration, to hydraulically open the third conduit (333). The presence of the first valve (50), in particular, in the case of a single access vascular access device, and of the first and second valve 50, in the case of a double access, advantageously enable limiting the blood losses once the haemodialysis has ended, improving the tolerability thereof by the patient. The valve (50) advantageously stops the blood flow as soon as the cannula of the single-use device (102) of collecting and/or injecting blood according to the invention is extracted from the first conduit or from the second conduit at the end of dialysis. In this way it is possible to minimise blood loss from the patient subjected to dialysis, in particular when the patient is medicated with a fistula pressure pad on the vascular access comprising silver as described in patent documents EP2296717 and ITBO20080303, marketed by EMODIAL S.r.l.

The first and/or second valve (50) is preferably arranged, respectively, in the second conduit (3, 33) and/or in the third conduit (333) distally of the second end of the conduits. This enables using the vascular access healing device of the invention by inserting it at the vascular access point of the body and in the second conduit and/or in the third conduit to heal the valve access about the cylinder of the healing device as mentioned in the foregoing.

The first and/or the second valve (50) is advantageously configured to enable entry into the relative conduit (3, 233, 33) through the relative second end (21, 232) of the relative tubular element (2, 203, 303) of the first end (108) of the medical cannula (103) of the single-use device (102) of collecting and/or injecting.

In alternative preferred embodiments of the vascular access device for haemodialysis with single and double access, the first valve (50) is arranged between second end (21, 232) of the second tubular element (2, 203) and the first plate (4), wherein the first plate (4) comprises, at the relative first face, first engaging means (41) engaged with the second end (21, 232) of the second tubular element (203) and/or, when the third tubular element (303) is present, the second plate (304) and the second valve (50), the second valve (50) is arranged between the second end (332) of the third tubular element (303) and the second plate (304), the second plate (304) comprising, at the relative first face, second engaging means (41) for engaging in the second end (21, 232) of the third tubular element (303) (See FIGS. 9A-10F).

In preferred embodiments of the vascular access device for haemodialysis, both single and double access, the first valve (50) is arranged on the second face of the first plate (4), opposite the relative first face and comprises third engaging means (54) for friction engagement with fourth engaging means (55) predisposed in the second end (21, 232) of the second tubular element (203) and/or in the first plate (4), and, when the third tubular element (303), the second plate (304) and the second valve (50) are present, the second valve (50) is arranged on the second face of the second plate (304), opposite the relative first face and comprises fifth engaging means (54) for friction engagement with sixth engaging means (55) predisposed in the second end (332) of the third tubular element (303) and/or in the second plate (304) (see FIGS. 11A-14E). The first engaging means (41), and/or the second engaging means (41), and/or the third engaging means (54), and/or the fifth engaging means (54), can comprise a relative plurality of engaging elements (41, 54) projecting from the first face by friction engagement with the second end (21, 232).

It is however preferable, as shown in the figures of the vascular access device for haemodialysis with single and double access, for the first valve (50) and/or the second valve (50), when present, to comprise a relative through-slot (56) which identifies two operating edges, wherein, in the relative closed configuration of the first and/or the second valve (50), the operating edges are in contact to prevent passage of fluids through the valve (50), and wherein the first and/or second valve (50) is elastically deformable to obtain the relative open configuration wherein the operating edges are moved away from one another to enable passage of a fluid through the slot (50) (see FIGS. 18A-18N, in particular 18F). In a preferred aspect of the invention in the vascular access device for haemodialysis, both with single and double access, the first valve (50) and/or the second valve (50), when present, can comprise an elastically deformable element which, in the relative closed configuration, is not deformed and obstructs, respectively, the relative second conduit 33, 233 and/or the relative third conduit 333 and in a relative open configuration is compressed and does not obstruct the relative second conduit (33, 233) and/or the relative third conduit (333).

In alternative preferred embodiments of the vascular access device for haemodialysis with single and double access, the first valve (50) has a relative perimeter and is arranged in the second conduit (33, 233) at the first end (31, 231) of the second tubular element (3, 203) with a first portion (151) of the relative perimeter fixed to the second conduit (33, 233) and with a remaining second portion (152) of the relative perimeter which is free, and which in the relative closed configuration, faces and is in contact with the second conduit (3, 233) in order to hydraulically close the second conduit and/or (preferably and), when present, the third tubular element (303), the second plate (304) and the second valve (50), the last having a relative perimeter and being arranged in the third conduit (333) at the first end (332) of the third tubular element (303) with a first portion (151) of the relative perimeter fixed to the third conduit (333) and with a remaining second portion (152) of the relative perimeter which is free, and which in the relative closed configuration, faces and is in contact with the second conduit (233) in order to hydraulically close the second conduit (233); and wherein the first and/or second valve (50) is elastically deformable to obtain the relative open configuration wherein the relative second portion of the relative perimeter is not in contact with respectively the second conduit (3, 233) and/or with the third conduit (333) to hydraulically open, respectively the second conduit (3, 233) and/or with the third conduit (333) (see FIGS. 19A-19M in particular 19F and 19G).

In a particularly preferred aspect of the invention the vascular access device for haemodialysis of the double access invention, which thus comprises the third tubular element (303), further comprises: a first and a second contact surface (401, 402) (See FIGS. 18D, 18N, 19E and 19N) which originate respectively from the first and second end (221, 223) of the first tubular element (202), wherein each of the first and second contact surface (401, 402) is configured to partially face a transversal section of an outer wall of blood vessel (A, V) in which a surgical incision has been made, completely surrounding the surgical incision, in order to facilitate the fixing, respectively, of the first and second end (221, 223) of the first tubular element (202) to the wall with the aim of hydraulically connecting the relative surgical incision to the first conduit (222). In this case, in the above-mentioned surgical method, the step of predisposing the vascular access device (1) for haemodialysis includes the double access vascular access device (1) has the first and second contact surface (401, 402) and the steps of fixing the first or second end (231, 232) of the first tubular element (202) to the vein and to the artery can advantageously comprise the positioning of the first and second contact surface (401, 402) at a transversal section, respectively of the vein and the artery facing the relative transversal sections and completely surrounding the relative surgical incision. Further, in this case, according to a particularly preferred aspect of the invention, each of the first and second contact surface (401, 402) comprises a fourth plurality of through-holes (48) (See FIGS. 19E and 1M) predisposed to surround the surgical incision to allow the fixing by means of corresponding suture stitches passing through the relative plurality of through-holes of the first and second contact surface (401, 402) (See FIGS. 18D, 18N, 19E and 19N) and through the wall of the blood vessel (A, V). Alternatively, it is preferable for each of the first and second contact surface (401, 402) (See FIGS. 18D, 18N, 19E and 19N) to be continuous surfaces, smooth and/or knurled, to facilitate the fixing thereof using surgical glue to the wall of the blood vessel (A, V). In this case, in the above-mentioned surgical method, and the step of fixing the first or second end (231, 232) of the first tubular element (202) to the vein and to the artery can advantageously include gluing, using surgical glue, the first and second contact surface (401, 402), respectively, to the transversal section, respectively of the vein and the artery while the first and second contact surface (401, 402) face them and completely surround the relative surgical incision.

With the aim of maximising the collection and injection velocity of blood during haemodialysis a particular preference is for both sterile packages and a collection and/or injection system (1000) for haemodialysis, according to the invention, wherein the first conduit (22) and the second conduit (33) have a relative minimum internal diameter and the cannula has a relative external diameter that is smaller by 0.2-0.3 mm than the smallest between the minimum internal diameters of the first conduit (22) and the second conduit (33). This enables, in fact, collection and/or injection of a greater blood flow.

For the purpose of maintaining sterility it is preferable that, in both the sterile package of the invention and in the collection and/or injection system for haemodialysis of the invention, the access element has a relative connection end which is: distal to the first end (108) of the medical cannula (103) and is engageable with a needle-free syringe, wherein the access element further comprises an elastically deformable element arranged in the access channel (181), and wherein the access channel (181) and the elastically deformable element are configured and mutually arranged in such a way that, when the connection end is disengaged, the elastically deformable element is not deformed and obstructs the access channel (181), in order to prevent a passage of liquids and aerosols, in such a way that, when the connection end is engaged with the needle-free syringe, the elastically deformable element is compressed by the needle-free syringe and de-obstructs the access channel (181) in order to hydraulically connect the syringe to the first end (108) of the medical cannula (103). In the package and the system the access element enables insertion of any anticoagulants into the access device, once implanted while the closing means (180) can be constituted by or can comprise a cap or lid, preferably friction-coupled, or a valve. The access element, when the first gripping means are present, can be arranged at the first gripping means (120) between the first gripping means (120) and the regulating and/or intercepting device (104) (see FIG. 5C). The first and/or the second gripping means can be constituted by a typical “butterfly” element that is usually applied to the medical cannulas.

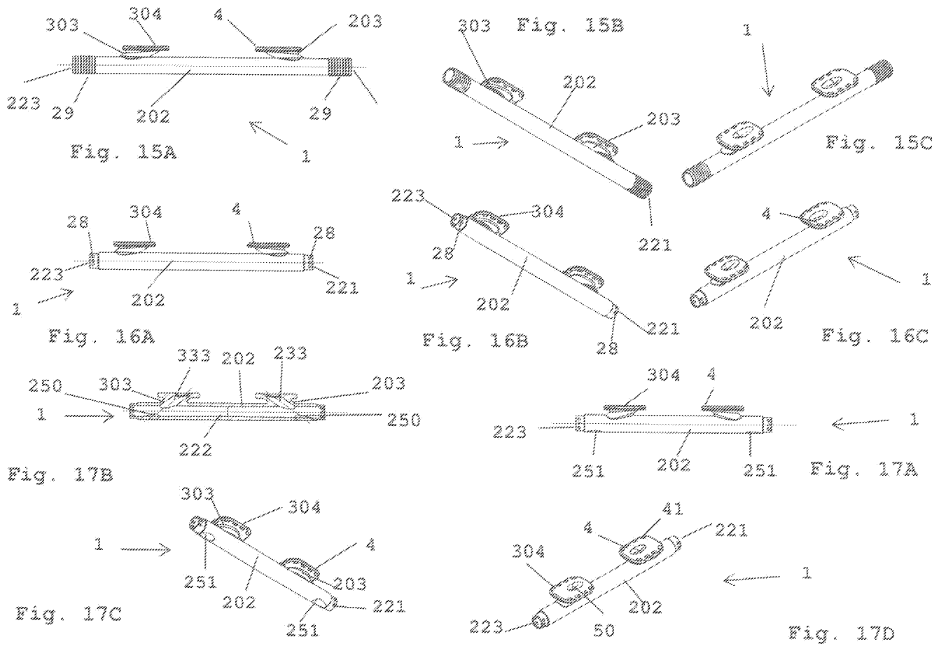

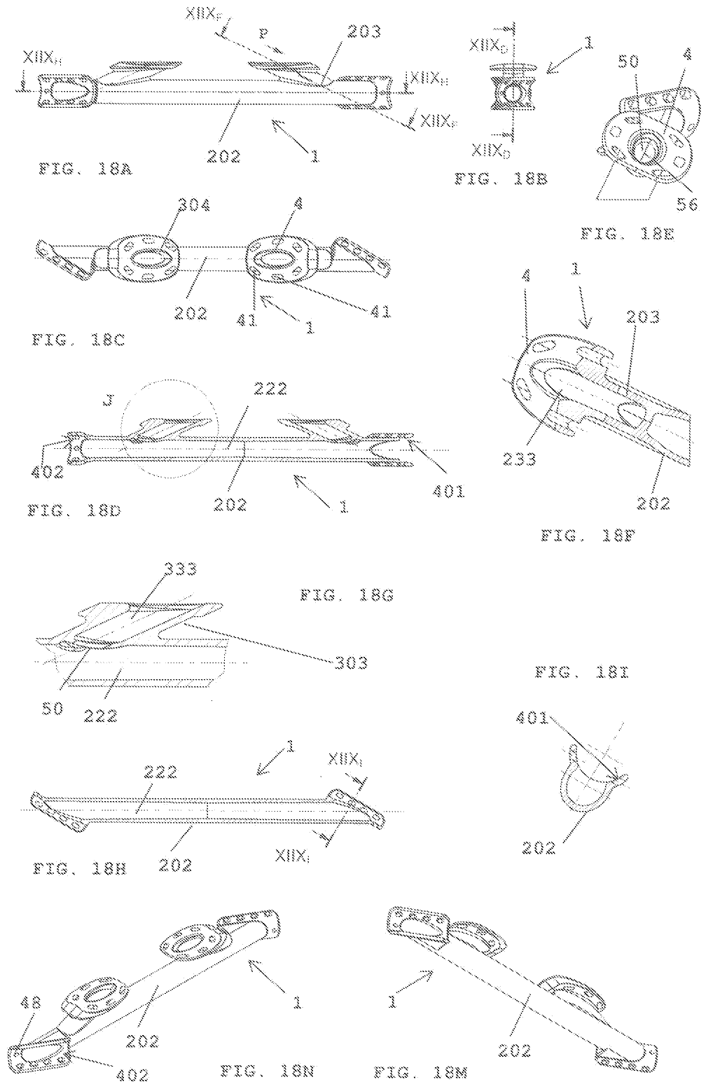

To facilitate the insertion in the correct side of the first conduit (2, 202), the first end (108) of the medical cannula (103) through the second conduit (33, 203) and the first conduit (22, 222) or through the third conduit (333) and the first conduit (22, 222) of a vascular access device (1) the first conduit (22, 222) can comprise a first and/or a second protuberance (250) arranged respectively on the side opposite the second (33, 203) and third conduit (333), between, respectively, the second conduit (33, 202) and the first end (221) of the first tubular element (2, 202) and the third conduit (333), and the second end (223) of the first tubular element (202) and conformed so as to enable the first end (108) of the medical cannula (103) to pass over the protuberance and proceed respectively towards the first and second end (221, 223) of the first tubular element (202). To obtain the protuberances (250) it is possible to deform the first tubular element (202) from outside obtaining corresponding concavities (251) on the opposite side to the second and/or third tubular element (3, 202, 303) (See FIGS. 17A-17C).

The system preferably also comprises a syringe containing a pharmaceutically injectable anticoagulant liquid composition, and more preferably packaged in a syringe, advantageously a needle-free syringe.

It is understood that the foregoing is described by way of non-limiting example, and that any variants of a practical-applicational nature are taken to fall within the protective scope of the invention as described in the foregoing and as claimed in the following.

1. A vascular access device for haemodialysis permanently implantable in a patient having an arteriovenous fistula, the vascular access device being made of a biocompatible and sterilisable material, and comprising:
a relative first tubular element having a first and a second relative end, which are longitudinal, the first tubular element defining a first conduit which extends from the first to the second end;
a relative second tubular element having a first and a second relative end which are longitudinal, and defining a second conduit which extends from the relative first to the second end; wherein the first end of the second tubular element is fixed to the first tubular element in an intermediate position between the first and the second end of the first tubular element, so that the second conduit is in fluid communication with the first conduit and so that the second end of the second tubular element is on a first side with respect to the first tubular element; and
first fixing means comprising a first plurality of through-holes and being fixed to the first tubular element and/or second tubular element the first plurality of through-holes being arranged on the first side, with respect to the first tubular element, and at the second end of the second tubular element, wherein said vascular access device is conformed and dimensioned to be implantable in an arm of a patient having an arteriovenous fistula for haemodialysis with the first conduit interposed in series to the arteriovenous fistula, with respect to the blood flow therein, between a first portion and a second portion of the arteriovenous fistula, the first end and the second end of the first tubular element being fixed, respectively to the first portion and to the second portion of the arteriovenous fistula, at a relative end of the relative portion and the second end of the second tubular element and with the first plurality of through-holes arranged subcutaneously at an implant skin region in order to enable fixing of the first plurality of through-holes to the implant skin region by means of a first plurality of suture stitches, and a consequent fixing of the second end of the second conduit to the implant skin region.

2. A vascular access device for haemodialysis permanently implantable in a patient, the vascular access device being made of a biocompatible and sterilisable material, and comprising:
a relative first tubular element having a first and a second relative end which are longitudinal, the first tubular element defining a first conduit which extends from the first to the second end;
a relative second tubular element having a first and a second relative end which are longitudinal, and defining a second conduit which extends from the relative first to the second end; wherein the first end of the second tubular element is fixed to the first tubular element in proximity of the first end of the first tubular element, in such a way that the second conduit is in fluid communication with the first conduit and so that the second end of the second tubular element is on a first side with respect to the first tubular element;
a relative third tubular element having a first and a second relative end which are longitudinal, third tubular element defining a third conduit which extends from the relative first to the second end; wherein the first end of the third tubular element is fixed to the first tubular element in proximity of the second end of the first tubular element, in such a way that the third conduit is in fluid communication with the first conduit and with the second conduit and so that the second end of the third tubular element is on the first side with respect to the first tubular element; and
first fixing means comprising a first plurality of through-holes and being fixed to the first tubular element and/or second tubular element, the first plurality of through-holes being arranged on the first side, with respect to the first tubular element, and at the second end of the second tubular element,
second fixing means comprising a second plurality of through-holes and being fixed to the first tubular element and/or third tubular element, the second plurality of through-holes being arranged on the first side, with respect to the first tubular element, and at the second end of the third tubular element,
wherein the vascular access device is conformed and dimensioned to be implanted in a patient in:
a first position which is at an artery, at a vein, or at a pre-existing arteriovenous fistula the first conduit being interposed in series with respect to the blood flow, respectively in the artery, in the vein or in the pre-existing arteriovenous fistula between a first portion and a second portion, respectively, of the artery, vein, or pre-existing arteriovenous fistula, with the first end and the second end of the first tubular element fixed respectively to the first portion and second portion, and/or
in a second position, between the artery and the vein, with the relative first or second end of the first tubular element fixed to the artery and with the remaining one of the first or second relative end of the first tubular element, which is not fixed to the artery, fixed to the vein in such a way that the first conduit hydraulically connects the artery to the vein to constitute an artificial arteriovenous fistula,
in which, in the first and second position, the second end of the second tubular element and the second end of the third tubular element, the first and second plurality of through-holes are arranged subcutaneously in an implant skin region for implant in order to enable fixing of the first and second plurality of through-holes to the implant skin region by means of a corresponding first and a corresponding second plurality of suture stitches, and the consequent fixing, respectively, of the second end of the second conduit and the second end of the third tubular element to the implant skin region.

3. The vascular access device for haemodialysis of claim 2, wherein the first fixing means originate from the second end of the second tubular element and/or wherein, when present, the second fixing means originate from the second end of the third tubular element; wherein the first fixing means comprise: a plate having a relative main through-hole, wherein the first plurality of through-holes is arranged peripherally with respect to the main through-hole, wherein the plate is fixed at a relative first face to the second end of the second tubular element in such a way that the second conduit is accessible from the relative main through-hole; and/or wherein the second fixing means, when present, comprise a second plate having a relative main through-hole, wherein the second plurality of through-holes is arranged peripherally with respect to the relative main through-hole wherein the second further plate is fixed at a relative first face to the second end of the third tubular element so that the third conduit is accessible from the relative main through-hole.

4. The vascular access device for haemodialysis of claim 3, wherein the plate is fixed to the second tubular element by a relative second face to the second end opposite the relative first face and arranged along a plane that is parallel to the longitudinal development axis of the first tubular element and/or wherein, when present, the second plate is fixed to the third tubular element by a relative second face opposite the relative first face and arranged along a plane that is parallel to the longitudinal development axis of the first tubular element.

5. The vascular access device for haemodialysis of claim 2, further comprising a first valve connected to the second conduit and configured so as, in a relative closed configuration, to hydraulically close the second conduit and so as, in a relative open configuration, to hydraulically open the second conduit and/or, when the relative third tubular element is present, and a second valve connected to the third conduit and configured so as, in a relative closed configuration, to hydraulically close the third conduit and so as, in a relative open configuration, to hydraulically open the third conduit.

6. The vascular access device for haemodialysis of claim 5, wherein the first valve and/or the second valve comprises a relative through-slot which identifies two operating edges, wherein, in the relative closed configuration of the first and/or the second valve, the operating edges are in contact to prevent passage of fluids through the valve, and wherein the first and/or second valve is elastically deformable to obtain the relative open configuration wherein the operating edges are moved away from one another to enable passage of a fluid through the slot.

7. The vascular access device for haemodialysis of claim 5, wherein the first valve has a relative perimeter and is arranged in the second conduit at the first end of the second tubular element with a first portion of the relative perimeter fixed to the second conduit and with a remaining second portion of the relative perimeter which is free, and which, in the relative closed configuration, faces and is in contact with the second conduit in order to hydraulically close the second conduit and/or wherein, when present, the second valve has a relative perimeter and is arranged in the third conduit at the first end of the third tubular element with a first portion of the relative perimeter fixed to the third conduit and with a remaining second portion of the relative perimeter which is free, and which in the relative closed configuration, faces and is in contact with the second conduit in order to hydraulically close the second conduit; and wherein the first and/or second valve is elastically deformable to obtain the relative open configuration wherein the relative second portion of the relative perimeter is not in contact with, respectively, the second conduit and/or with the third conduit in order to hydraulically open, respectively, the second conduit and/or the third conduit.

8. The vascular access device for haemodialysis of claim 2, further comprising: a first and a second contact surface which originate respectively from the first and second end of the first tubular element, wherein each of the first and second contact surface is configured to partially face a transversal section of an outer wall of blood vessel in which a surgical incision has been made completely surrounding the surgical incision, in order to facilitate the fixing, respectively, of the first and second end of the first tubular element to the wall with the aim of hydraulically connecting the relative surgical incision to the first conduit.

9. The vascular access device for haemodialysis of claim 8, wherein each of the first and second contact surface comprise a fourth plurality of through-holes predisposed to surround the surgical incision to allow the fixing by means of corresponding suture stitches through the relative plurality of through-holes of the first and second contact surface and through the wall of the blood vessel.

10. A healing device of a vascular access, comprising:
a relative insertion portion which is insertable internally of a vascular access and which comprises: a cylinder having a relative longitudinal development axis: a first and a second relative end which are longitudinal; and a distally tapered tip fixed to the first end of the cylinder; wherein the insertion portion is insertable in the second conduit or in the third conduit of a vascular access device for haemodialysis according to claim 5 from the relative second end so as to occlude exclusively a portion of the second conduit or the third conduit comprised between, respectively, the first valve and the first plate or between the second valve and the second plate;
a relative connection portion fixed to the second end of the cylinder; and
a relative first and a relative second gripping tab, fixed to the connection portion on opposite sides thereof and on opposite sides with respect to the cylinder axis and with possibility of relative movement with respect to the connection portion in order to assume: a first relative configuration in which the first and the second gripping tab are substantially coplanar; and a second configuration in which the tabs are opposite one another, wherein in both the first and the second configuration the first and the second gripping tab are not insertable in the second conduit or third conduit.

11. The healing device of a vascular access of claim 10, wherein the relative cylinder is inclined with respect to the connection portion by an angle identical to the angle with which the second tubular element or the third tubular element is inclined with respect, respectively, to the first plate or the second plate.

12. A collection and/or injection system for haemodialysis, comprising:
a vascular access device for haemodialysis of claim 2 and at least a single-use device of collecting and/or injecting blood in an arteriovenous fistula, a vein or an artery constituted by:
a medical cannula for cannulation having a relative first end and a relative second end wherein the first end is free and cannulable in a pre-existing arteriovenous fistula, an artery, or a vein of a patient through the second conduit and the first conduit or through the third conduit and the first conduit of a vascular access device;
a flow regulating and/or intercepting device predisposed to regulate and/or interrupt a flow of a liquid in the medical cannula;
a connecting element, fixed to the second end of the medical cannula to enable hydraulic connection to an inlet tube of blood or to an outlet tube of blood of a haemodialysis apparatus;
optionally an access element arranged between the first end of the cannula and the regulating and/or intercepting device and defining an access channel hydraulically connected to the first end of the cannula; and reversible closing means for reversibly closing the access channel at a distal end of the access element in order to enable injection of a liquid into the access channel and to cause the liquid to flow towards the first end of the medical cannula; and
optionally a first gripping element, arranged between the first end and/or the regulating and/or intercepting device, and/or a second gripping element, arranged proximally to the connecting element;
wherein the collection and/or injection system comprises optionally at least one healing device.

13. A sterile package containing at least a single-use device of collecting and/or injecting blood in an arteriovenous fistula constituted by:
a medical cannula for cannulation having a relative first end and a relative second end wherein the first end is free and cannulable in a pre-existing arteriovenous fistula, an artery, or a vein of a patient through the second conduit and the first conduit or through the third conduit and the first conduit of a vascular access device according to claim 2;
a flow regulating and/or intercepting device predisposed to regulate and/or interrupt a flow of a liquid in the medical cannula; and
a connecting element, fixed to the second end of the medical cannula to enable hydraulic connection to an inlet tube of blood or to an outlet tube of blood of a haemodialysis apparatus;
optionally an access element arranged between the first end of the cannula and the regulating and/or intercepting device and defining an access channel hydraulically connected to the first end of the cannula; and reversible closing means for reversibly closing the access channel at a distal end of the access element in order to enable injection of a liquid into the access channel and to cause the liquid to flow towards the first end of the medical cannula;
optionally a first gripping element, arranged between the first end and/or the regulating and/or intercepting device, and/or a second gripping element, arranged proximally to the connecting element.

14. The sterile package of claim 13, wherein the access element has a relative connection end which is: distal to the first end of the medical cannula and is engageable with a needle-free syringe, wherein the access element further comprises an elastically deformable element arranged in the access channel, and wherein the access channel and the elastically deformable element are configured and mutually arranged in such a way that, when the connection end is disengaged, the elastically deformable element is not deformed and obstructs the access channel, in order to prevent a passage of liquids and aerosols, in such a way that, when the connection end is engaged with the needle-free syringe, the elastically deformable element is compressed by the needle-free syringe and de-obstructs the access channel in order to hydraulically connect the syringe to the first end (108) of the medical cannula (103).

15. The vascular access device for haemodialysis of claim 1, wherein the first fixing means originate from the second end of the second tubular element and/or wherein, when present, the second fixing means originate from the second end of the third tubular element; wherein the first fixing means comprise: a plate having a relative main through-hole, wherein the first plurality of through-holes is arranged peripherally with respect to the main through-hole, wherein the plate is fixed at a relative first face to the second end of the second tubular element in such a way that the second conduit is accessible from the relative main through-hole; and/or wherein the second fixing means, when present, comprise a second plate having a relative main through-hole, wherein the second plurality of through-holes is arranged peripherally with respect to the relative main through-hole wherein the second further plate is fixed at a relative first face to the second end of the third tubular element so that the third conduit is accessible from the relative main through-hole.

16. The vascular access device for haemodialysis of claim 1, further comprising a first valve connected to the second conduit and configured so as, in a relative closed configuration, to hydraulically close the second conduit and so as, in a relative open configuration, to hydraulically open the second conduit and/or, when the relative third tubular element is present, and a second valve connected to the third conduit and configured so as, in a relative closed configuration, to hydraulically close the third conduit and so as, in a relative open configuration, to hydraulically open the third conduit.

17. A healing device of a vascular access, comprising:
a relative insertion portion which is insertable internally of a vascular access and which comprises: a cylinder having a relative longitudinal development axis: a first and a second relative end which are longitudinal; and a distally tapered tip fixed to the first end of the cylinder; wherein the insertion portion is insertable in the second conduit or in the third conduit of a vascular access device for haemodialysis according to claim 8 from the relative second end so as to occlude exclusively a portion of the second conduit or the third conduit comprised between, respectively, the first valve and the first plate or between the second valve and the second plate;
a relative connection portion fixed to the second end of the cylinder; and
a relative first and a relative second gripping tab, fixed to the connection portion on opposite sides thereof and on opposite sides with respect to the cylinder axis and with possibility of relative movement with respect to the connection portion in order to assume: a first relative configuration in which the first and the second gripping tab are substantially coplanar; and a second configuration in which the tabs are opposite one another, wherein in both the first and the second configuration the first and the second gripping tab are not insertable in the second conduit or third conduit.

18. A collection and/or injection system for haemodialysis, comprising:
a vascular access device for haemodialysis of claim 1 and at least a single-use device of collecting and/or injecting blood in an arteriovenous fistula, a vein or an artery constituted by:
a medical cannula for cannulation having a relative first end and a relative second end wherein the first end is free and cannulable in a pre-existing arteriovenous fistula, an artery, or a vein of a patient through the second conduit and the first conduit or through the third conduit and the first conduit of a vascular access device;
a flow regulating and/or intercepting device predisposed to regulate and/or interrupt a flow of a liquid in the medical cannula;
a connecting element, fixed to the second end of the medical cannula to enable hydraulic connection to an inlet tube of blood or to an outlet tube of blood of a haemodialysis apparatus;
optionally an access element arranged between the first end of the cannula and the regulating and/or intercepting device and defining an access channel hydraulically connected to the first end of the cannula; and reversible closing means for reversibly closing the access channel at a distal end of the access element in order to enable injection of a liquid into the access channel and to cause the liquid to flow towards the first end of the medical cannula; and
optionally a first gripping element, arranged between the first end and/or the regulating and/or intercepting device, and/or a second gripping element, arranged proximally to the connecting element;
wherein the collection and/or injection system comprises optionally at least one healing device.

19. A sterile package containing at least a single-use device of collecting and/or injecting blood in an arteriovenous fistula constituted by:
a medical cannula for cannulation having a relative first end and a relative second end wherein the first end is free and cannulable in a pre-existing arteriovenous fistula, an artery, or a vein of a patient through the second conduit and the first conduit or through the third conduit and the first conduit of a vascular access device according to claim 1;
a flow regulating and/or intercepting device predisposed to regulate and/or interrupt a flow of a liquid in the medical cannula; and
a connecting element, fixed to the second end of the medical cannula to enable hydraulic connection to an inlet tube of blood or to an outlet tube of blood of a haemodialysis apparatus;
optionally an access element arranged between the first end of the cannula and the regulating and/or intercepting device and defining an access channel hydraulically connected to the first end of the cannula; and reversible closing means for reversibly closing the access channel at a distal end of the access element in order to enable injection of a liquid into the access channel and to cause the liquid to flow towards the first end of the medical cannula;
optionally a first gripping element, arranged between the first end and/or the regulating and/or intercepting device, and/or a second gripping element, arranged proximally to the connecting element.
